(19) United States
(12) Patent Application Publication
(10) Pub. No.: US 2021/0283273 A1
(43) Pub. Date: Sep. 16, 2021

(54) NON-VIRAL DELIVERY AGENTS FROM POLYELECTROLYTES BASED ON CYCLOPROPENIUM IONS, THEIR SYNTHESES, AND USES THEREOF

(71) Applicant: THE TRUSTEES OF COLUMBIA UNIVERSITY IN THE CITY OF NEW YORK, New York, NY (US)

(72) Inventors: Luis M. CAMPOS, New York, NY (US); Tristan Hayes LAMBERT, Monroe, NY (US); Jessica L. FREYER, New York, NY (US); Spencer C. BRUCKS, Brooklyn, NY (US)

(21) Appl. No.: 16/326,606

(22) PCT Filed: Aug. 18, 2017
(86) PCT No.: PCT/US17/47562

§ 371 Date: Feb. 19, 2019

Related U.S. Application Data
(60) Provisional application No. 62/377,491, filed on Aug. 19, 2016.

(51) Int. Cl. A61K 48/00; A61K 47/59; A61K 47/54; A61K 47/58; C08F 12/26; C08F 293/00; C08G 73/02; C08F 8/32; C08G 81/00; C12N 15/87

(52) U.S. Cl. CPC A61K 48/0041; A61K 47/59; A61K 47/541; A61K 47/58; C08F 12/26; C08F 2438/01; C08G 73/0206; C08F 8/32; C08G 81/00; C12N 15/87; C08F 293/005

(57) ABSTRACT

Non-viral gene delivery agents can be provided comprising a polyamine comprising a polymer containing a secondary amine, wherein the polyamine and a derivative of a cyclopropenium ion that are covalently attached. The non-viral gene delivery agents can be formed by a click reaction combining a polyamine polymer precursor backbone with a trisaminocyclopropenium polymer. The non-viral gene delivery agents can be used to deliver genetic material to cells in mammals or other organisms as part of gene therapy.

### CROSS-REFERENCE TO RELATED APPLICATION(S)

This application claims priority from U.S. Provisional Application No. 62/377,491, filed on Aug. 19, 2016, the entire contents of which is incorporated herein by reference.

### STATEMENT REGARDING FEDERALLY SPONSORED RESEARCH OR DEVELOPMENT

The present application was made with government support under contract number CAREER DMR-1351293 awarded by the National Science Foundation. The United States government may have certain rights in this application.

### FIELD OF THE DISCLOSURE

Aspects of the present disclosure relate to derivatized polymers for delivery systems, such as for example, gene delivery, the synthesis of such derivatized polymers, and to the use of such derivatized polymers that are non-cytotoxic in delivery systems.

### BACKGROUND INFORMATION

Delivery of genetic material for therapy or other purposes is commonly known, particularly for the treatment of diseases including certain cancers. Cell delivery systems may involve direct injection of naked DNA, use of viruses or genetically modified viruses, and those delivery methods that make use of non-viral delivery agents. Each of these systems has its advantages and disadvantages. Viruses as delivery agents are advantageous for their high efficiency and high cell selectivity; however, they are also disadvantageous due to toxicity, the production of inflammatory responses, and difficulty with the use of large DNA fragments.

Non-viral gene delivery systems are based on genetic material being encompassed by or complexed to particles by electrostatic interactions between the negatively charged phosphate backbone of DNA and cationic particles, including, for example, polymers, lipids, or peptides (Erbacher, P. et al, Gene Therapy, 1999, 6, 138-145). It has been suggested that complexes formed between a nucleic acid and a lipid attach to a cell surface, then pass into the cell by endocytosis. The complex is localized within a vesicle or endosome and the nucleic acid is released into the cytoplasm. Eventually, the nucleic acid migrates into the nucleus, where a gene encoded by the nucleic acid may be expressed. DNA is transcribed into RNA and then translated into protein.

Modular polyelectrolytes have the potential to be transformative in applications such as delivery systems, energy storage, electronic devices, and materials that possess both inherent compositional modularity and accessibility via robust and scalable synthetic pathways that are of particular import to these fields. To date, development of cationic polyelectrolytes has focused on a limited menu of monomers, most of which bear charge formally localized on heteroatoms and lack chemical handles to tune their physical properties (e.g. imidazolium, ammonium, and phosphonium).

The ability to control macromolecular architecture and synthetic tenability [see Ref. 1] of cationic building blocks has contributed to the widespread use of poly(ionic liquids) (PILs, FIG. 1A), or polyelectrolytes, in various applications [see Ref. 2] ranging from gene delivery vectors [see Ref 3] to alkaline fuel cells. [See Ref 4] Cationic PILs are being explored in applications ranging from medicine to energy storage, and thus it is highly desirable to develop efficient synthetic strategies to target innovative cationic building blocks. As the understanding of structure-property relationships concerning charge density, repeat unit composition, and macromolecular structure in such polymeric systems has developed, [see Ref 5] so too has the need for synthetic strategies to target new classes of these materials (FIG. 1B). [See Ref. 6] However, manipulating the functionality, processability, and Coulombic interactions of PILs presents a significant challenge, [see Ref. 7] and developing detailed structure-property relationships for cellular transfection applications has been limited. Chemical transformations that overcome such obstacles have the potential to broaden the fundamental understanding of polyelectrolytes in modern technologies, particularly gene-based therapies. [see Ref. 8]

Trisaminocyclopropenium (TAC) based polymers, where the formal charge is on carbon, but highly delocalized within the monomer (a soft cation) have been synthesized. [See Ref. 9] Initial structure-property relationships of functional TAC PILs with regard to ionic conductivity and processability underscored the necessity for an alternative synthetic strategy, as performing many polymerizations is cumbersome and polymers comprising different TAC derivatives had batch-to-batch variations. Akin to what Coates and co-workers have demonstrated with alkaline-stable imidazolium ionic liquids (ILs), the ability to elaborate cationic building blocks towards complex structures that are not commercially available is crucial to optimize performance for a given application. [See Ref 10]

Cationic polymers are among the most common non-viral gene delivery vectors because of their ability to complex with the negatively charged phosphate backbone of DNA, and the formation of these polyplexes can prevent degradation of genetic material and encourage cellular uptake (FIG. 1C). [See Refs. 8a, 11] However, if the electrostatic cohesion between polymer and DNA is too strong for adequate release of DNA into the cell, transfection efficiency can be dramatically suppressed. [see Ref. 12] In fact, Schmuck and co-workers have shown that the specific nature of the association between the cationic building block and the DNA, and the ability to manipulate these Coulombic interactions is instrumental for optimization of transfection efficiency. [See Ref. 13] It is therefore important to study how various types of building blocks affect transfection. [See Refs. 3b, 8d, 14] Considering that trisaminocyclopropenium ions are remarkably stable cations, that have been observed to only weakly associate with their counterions, [15] there is a continuing need to investigate how these moieties would behave as transfection agents. Furthermore, because the cyclopropenium cation is stable across a broad pH range, [see Ref 16] resulting polyplexes may be particularly robust. For these reasons, coupled with the acute control of macromolecular architecture and molecular structure this system permits, there is a need for the development of a post polymerization strategy towards TAC polymers that would serve as an effective platform to synthesize transfection agents.

The modification of polymer backbones with functional groups by modular and efficient chemistries, especially via “click” reactions, is particularly desirable for materials commercialization. [See Ref 17] The limited tolerance of myriad functional groups in controlled polymerization techniques (FIG. 1A) renders post-polymerization functionalizations (PPF, FIG. 1B) an attractive route to complex macromolecular structures of polyelectrolytes. [See Ref. 18] PPF is especially attractive for PILs, as charged groups are incompatible with most size exclusion chromatography (SEC) columns. As a result, many studies of PILs ignore effects of molecular mass and dispersity (Ð), correlating physical properties solely to the structure of repeat units. [See Ref. 19] A more complete understanding of macromolecular systems can be achieved in materials with well-defined and narrow molecular weight distributions. [See Ref. 20]

Thus, a further method to synthesize TAC-based polyelectrolytes to simultaneously control the macromolecular architecture and molecular composition of TAC repeat units, the resulting derivatized polymers for delivery systems, and their use are thus desired and needed. A post-polymerization click reaction that provides facile access to cyclopropenium (CP) ion-functionalized macromolecules of various architectures, including microphase segregated block copolymer membranes, is also desired. Therefore, straightforward access to a variety of amino substituents on the TAC scaffold could facilitate optimization, inform design principles, and elucidate chemical structure-property relationships within a single family of materials to improve performance in applications such as non-viral gene delivery.

### SUMMARY OF EXEMPLARY EMBODIMENTS

Exemplary aspects of the present application are directed to a novel non-viral delivery agent, that is non-cytotoxic and easily and quickly synthesized.

One exemplary embodiment of the present disclosure includes a non-viral gene delivery agent comprising: a polyamine comprising a polymer containing a secondary amine; a derivative of the cyclopropenium ion; and wherein the polyamine and the derivative of the cyclopropenium ion are covalently attached. In one embodiment, the derivative is a bis(dialkylamino)cyclopropenium chloride salt. In another embodiment, the secondary amine is a dialkylamino group. In certain embodiments, the dialkylamino group is selected from the group consisting of: dicyclohexylamine, diisopropylamine, morpholine, piperidine, diethylamine, diallylamine, dibutylamine, and combinations thereof. In certain embodiments, the polyamine is a linear polyethyleneimine or a linear polyvinylbenzylmethylamine or poly(methylaminostyrene). In further embodiments, the derivative of the cyclopropenium ion is bis(dialkylamino)cyclopropenium chloride (BACC1) ionic liquid.

In some exemplary embodiments of the present disclosure, the agent is selected from the group consisting of or comprising:

poly(methylaminostyrene)(piperidine), poly(methylaminostyrene)(morpholine), polyethyleneimine(piperidine), poly(methylaminostyrene)(n-butyl), poly(methylaminostyrene)(isopropyl), polyethyleneimine(n-butyl), polyethyleneimine(isopropyl), and polyethyleneimine(morpholine). In certain exemplary embodiments, the delivery agent has a no pH-dependent charge and a formal charge on a carbon atom.

Another exemplary embodiment of the present disclosure is directed to a non-viral transfection agent, where the agent comprises:

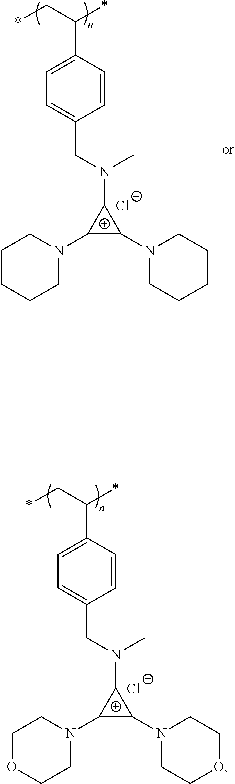

wherein n=100-200. In certain exemplary embodiments, n=130-140. In particular embodiments, n=135.

Another aspect of the present disclosure is a non-viral transfection agent of:

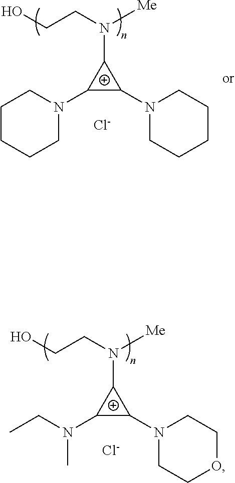

wherein n=220-590. In certain embodiments, n=575-585. In certain embodiments, n=581.

Another exemplary embodiment of the present disclosure is directed to a method of transfecting a cell with a genetic material, method of transfecting a cell with a genetic material, comprising the steps of: complexing a non-viral gene delivery agent with the genetic material to form a polyplex; administering the polyplex to a candidate set of cells for transfection; transfecting the cells with the polyplex; and where the non-viral gene delivery agent comprises a polyamine comprising a polymer containing a secondary amine, wherein the polyamine and a derivative of the cyclopropenium ion are covalently attached.

In certain exemplary embodiments of the present disclosure, the method can include a use of a transfection agent selected from the group consisting of or comprising: poly(methylaminostyrene)(piperidine), poly(methylaminostyrene)(morpholine), polyethyleneimine(piperidine), poly(methylaminostyrene)(n-butyl), poly(methylaminostyrene)(isopropyl), polyethyleneimine(n-butyl), polyethyleneimine(isopropyl) and polyethyleneimine(morpholine).

Another exemplary embodiment of the present disclosure is directed to a method of synthesis of a non-viral gene delivery agent comprising: polymerizing a polymer precursor backbone, wherein the polymer precursor backbone is a polyamine comprising a polymer containing a secondary amine; polymerizing a bis(dialkylamino)cyclopropenium chloride derivative (BACC1); covalently attaching the BACC1 to the polymer precursor backbone by a click reaction; and wherein the non-viral delivery agent produced by the click reaction is a trisaminocyclopropenium (TAC) polymer, and further wherein the TAC polymer comprises a polyamine comprising a polymer containing a secondary amine, where the polyamine and a derivative of the cyclopropenium ion are covalently attached.

These and other exemplary aspects and embodiments of the present disclosure will become better understood with reference to the following detailed description when considered in association with the accompanying drawings and claims.

### BRIEF DESCRIPTION OF THE DRAWINGS

FIG. 1 shows exemplary synthetic strategies to access polyelectrolytes by (A) polymerization of ionic liquids that contain a polymerizable unit; and (B) modification of the polymer backbone with a neutral group that yields charged moieties or by directly using a charged functional group to couple to the backbone. (C) PIL/pDNA polyplexes transfect cells and induce luciferase expression, resulting in cell luminescence.

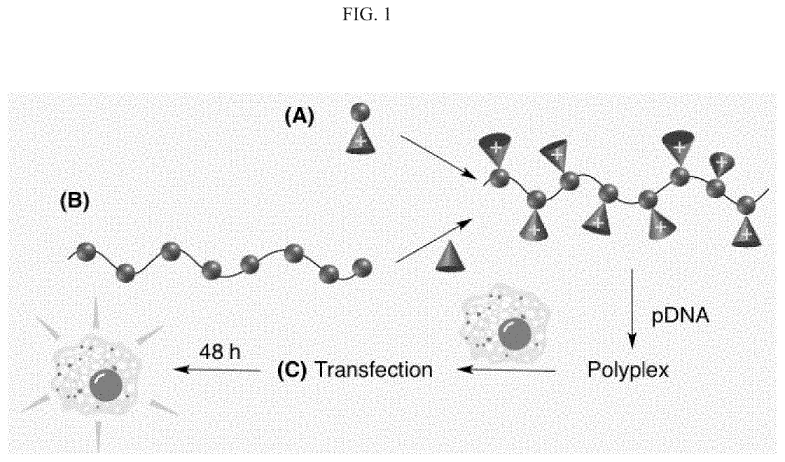

FIG. 2 shows 1H NMR spectra of TAC polymershaving complete functionalization of PMAS with BACCI ClickabILs bearing various alkyl substituents. (See, FIGS. 10-34 and Examples for full NMR spectra).

FIG. 3 shows exemplary post-polymerization functionalization of polymers containing secondary amines by the addition of BACC1 ClickabILs.

FIG. 4 shows cell viabilities of HEK-293T cells following 48 h incubation with TAC functionalized polymers at various doses. Error bars are standard deviation of triplicate measurement. Polyethyleneimine (PEI); Piperidine (Pip); Morpholine (Mo); Polystyrene-Piperidine (PS-Pip); Polystyrene-Morpholine (PS-Mo)

FIG. 5 shows Luciferase expression of transfected HEK-293T cells using TAC polymers and 25K linear PEI. Polymer backbones are noted in white boxes and amino substituents pictured above respective bars. Luciferase expression of cells is measured after 48 h incubation with specified polymer loadings (all with pDNA loading of 3 μg mL-1) and normalized by cell count. Error bars are standard deviation of triplicate measurement.

FIG. 6 shows an exemplary scheme of the synthesis of Poly(methylaminostyrene)(trisaminocyclopropenium) (PMAS(TAC)).

FIG. 7 shows GPC traces of polystyrene macroinitiator and PS-b-PBoc which reveal narrow dispersity that is maintained after copolymerization.

FIG. 8 shows GPC traces of poly(ethylene oxide) macroinitiator and PEO-b-PBoc which reveal narrow dispersity that is maintained after copolymerization.

FIG. 9 shows an exemplary synthetic path to obtain BACC1 ClickabIL building blocks.

FIG. 10 shows ¹H NMR spectrum of bis-1,2-(diallylamino)-3-chlorocyclopropenium chloride.

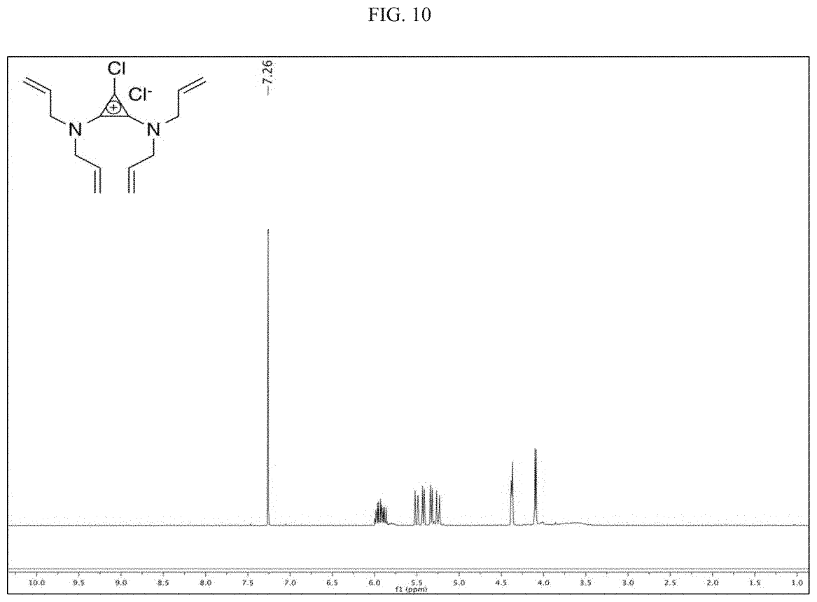

FIG. 11 shows ¹H NMR spectrum of bis-1,2-(piperidino)-3-chlorocyclopropenium chloride.

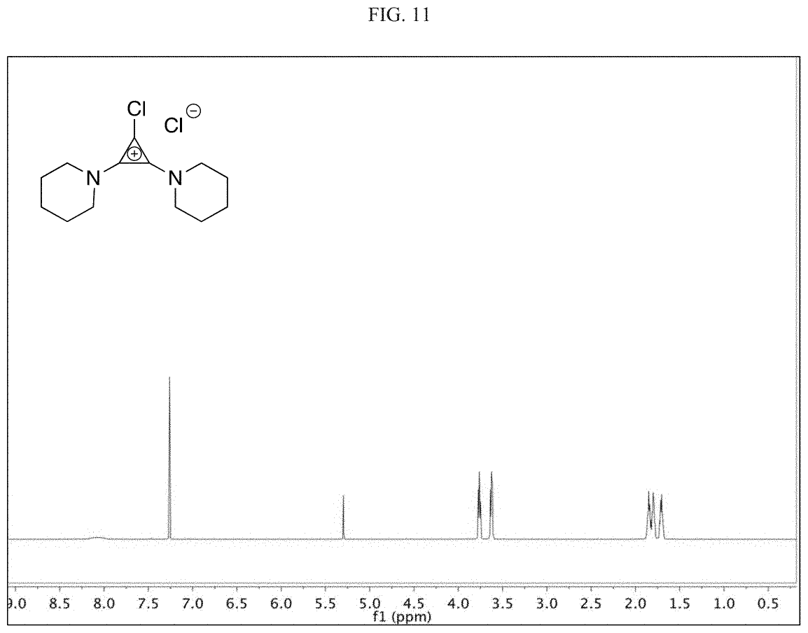

FIG. 12 shows ¹H NMR spectrum of tert-butyl methyl(4-vinylbenzyl)carbamate.

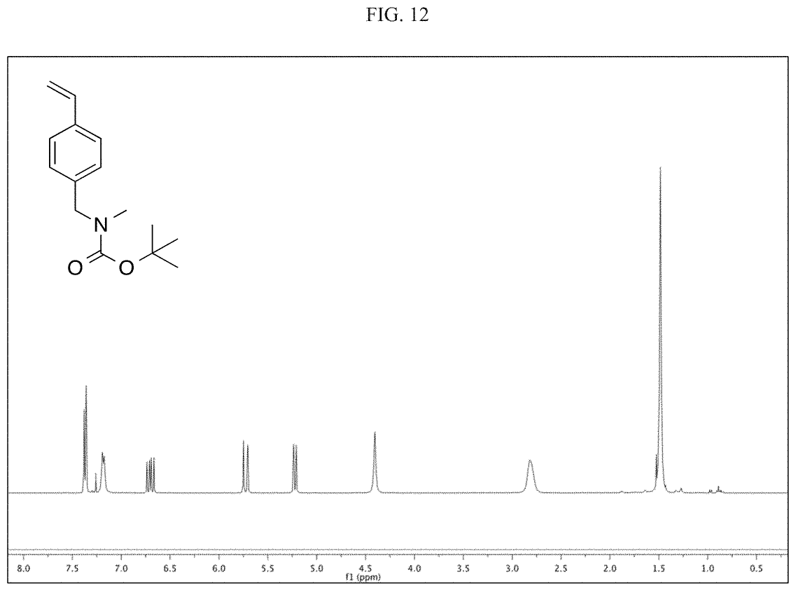

FIG. 13 shows ¹H NMR spectrum of PBoc.

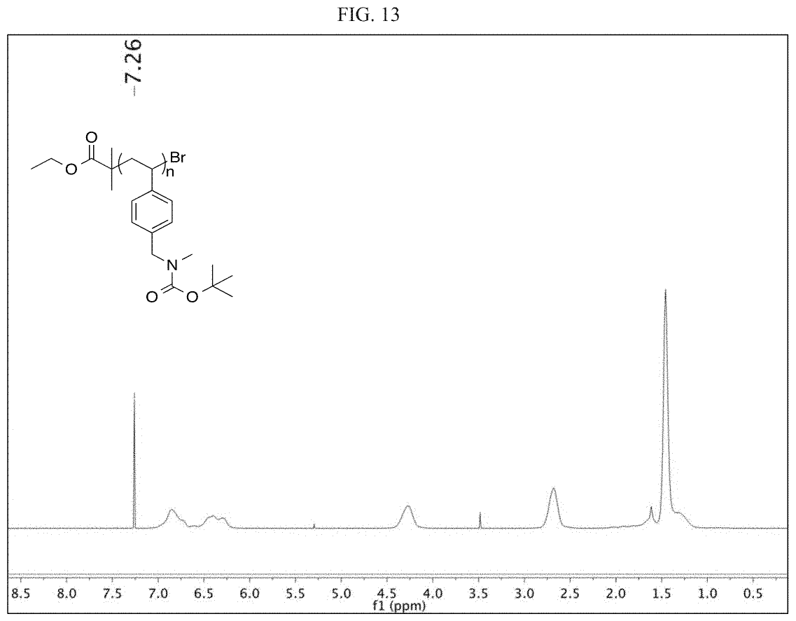

FIG. 14 shows ¹H NMR spectrum of PMAS.

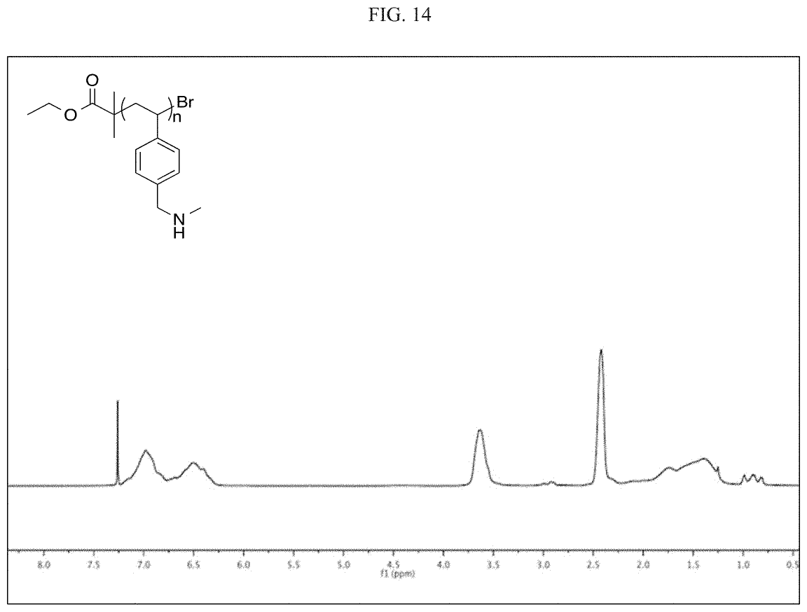

FIG. 15 shows ¹H NMR spectrum of PTACCy.

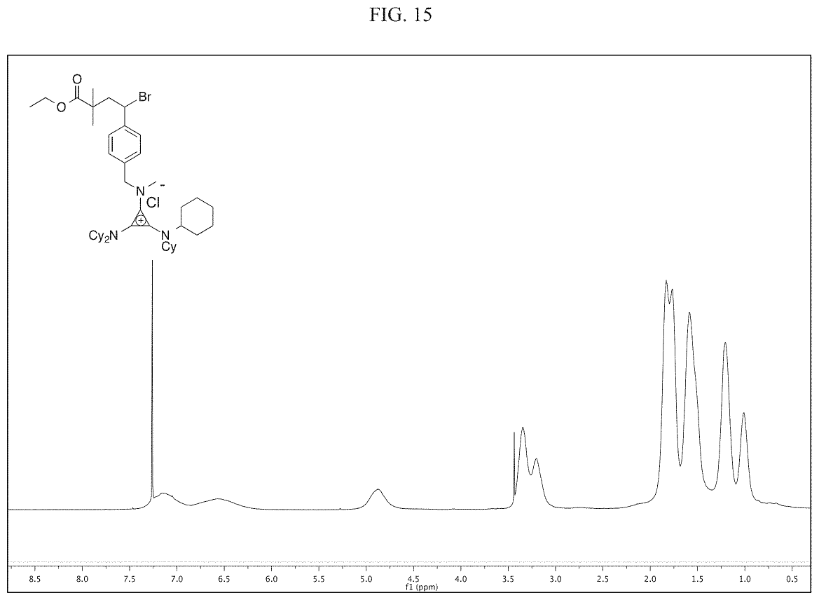

FIG. 16 shows ¹H NMR spectrum of PTACA1.

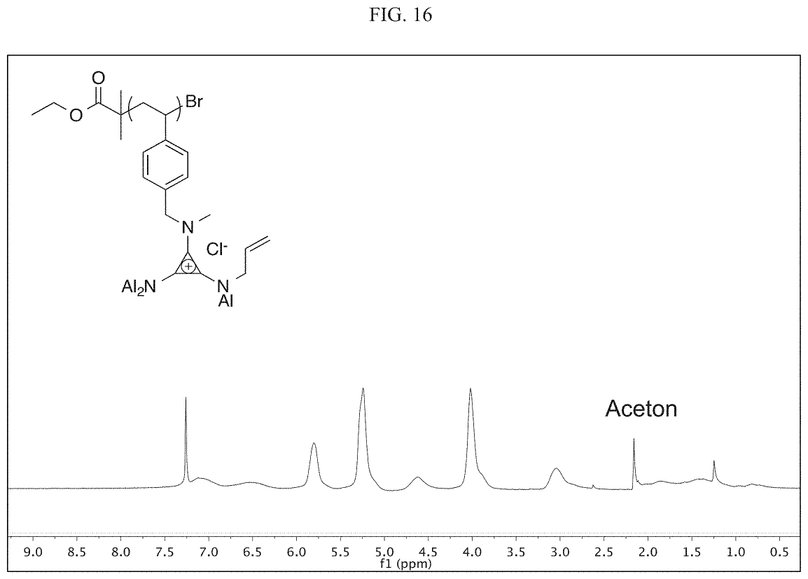

FIG. 17 shows ¹H NMR spectrum of PTACEt.

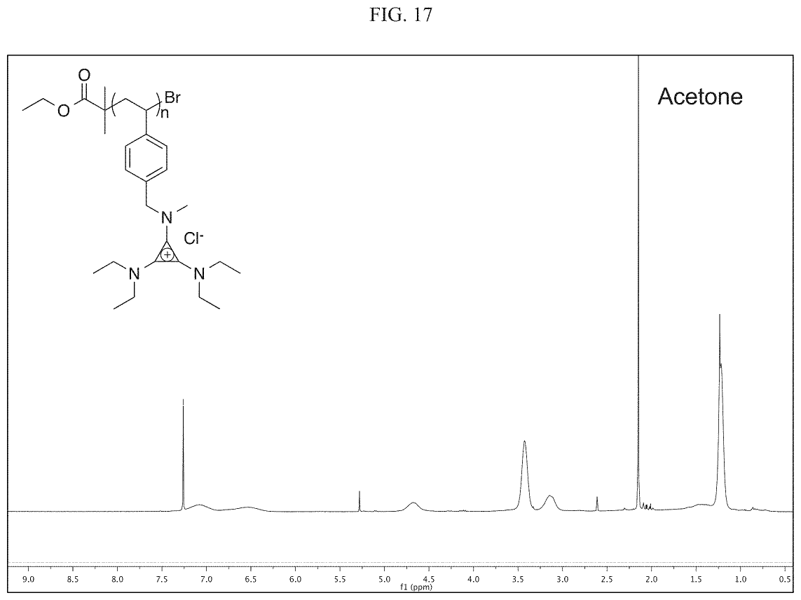

FIG. 18 shows ¹H NMR spectrum of PTACiP.

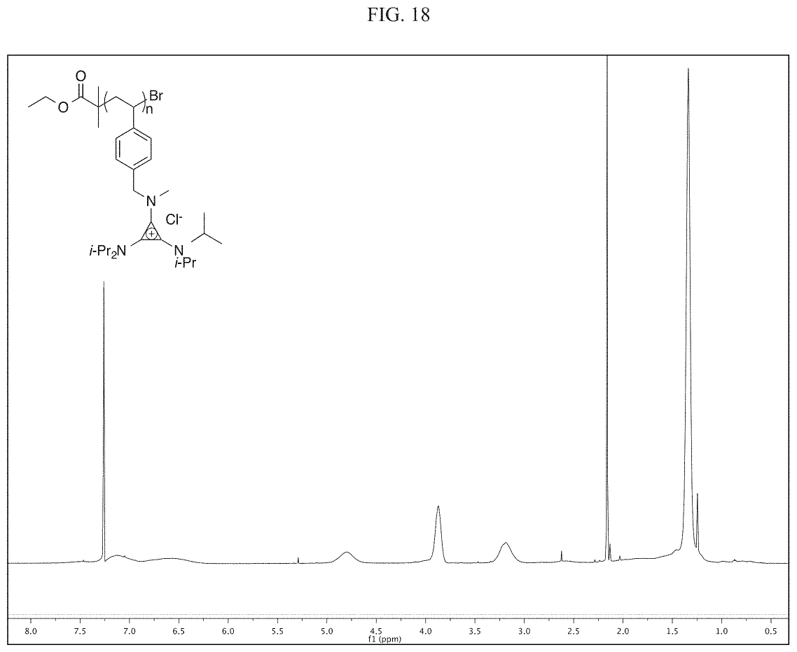

FIG. 19 shows ¹H NMR spectrum of PTACMo.

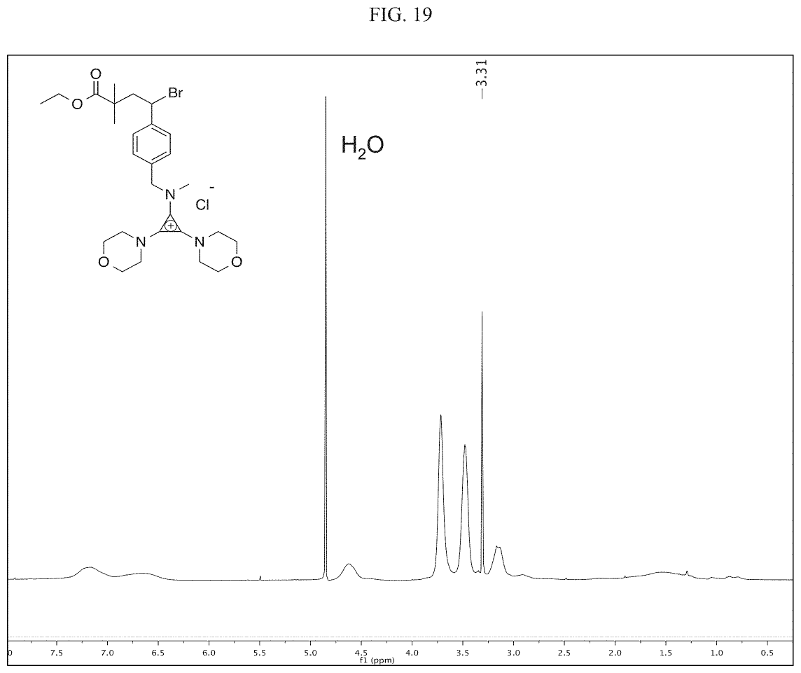

FIG. 20 shows ¹H NMR spectrum of PTACiP.

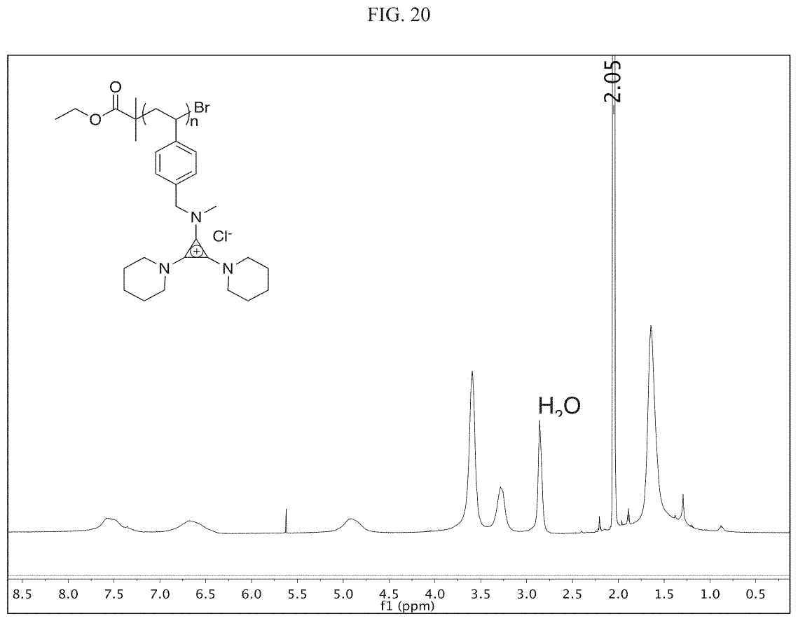

FIG. 21 shows ¹H NMR spectrum of PS-b-PBoc.

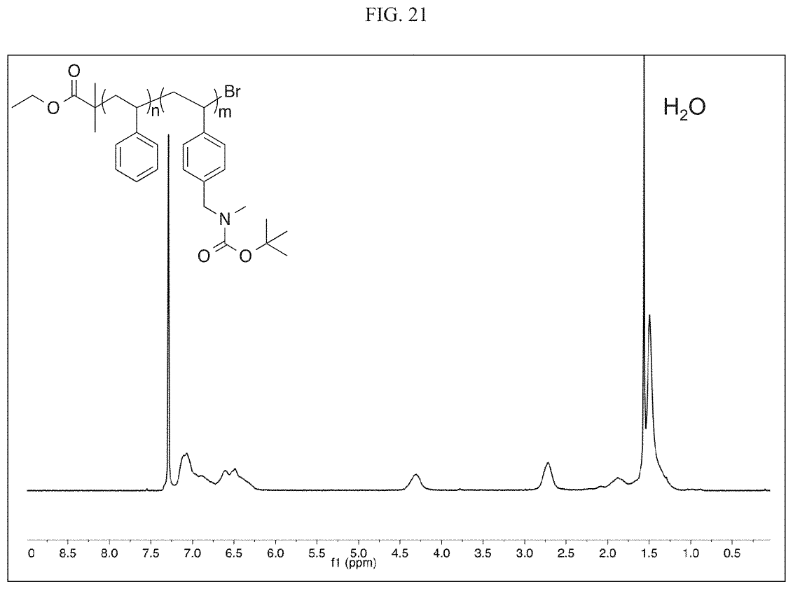

FIG. 22 shows ¹H NMR spectrum of PS-b-PMAS.

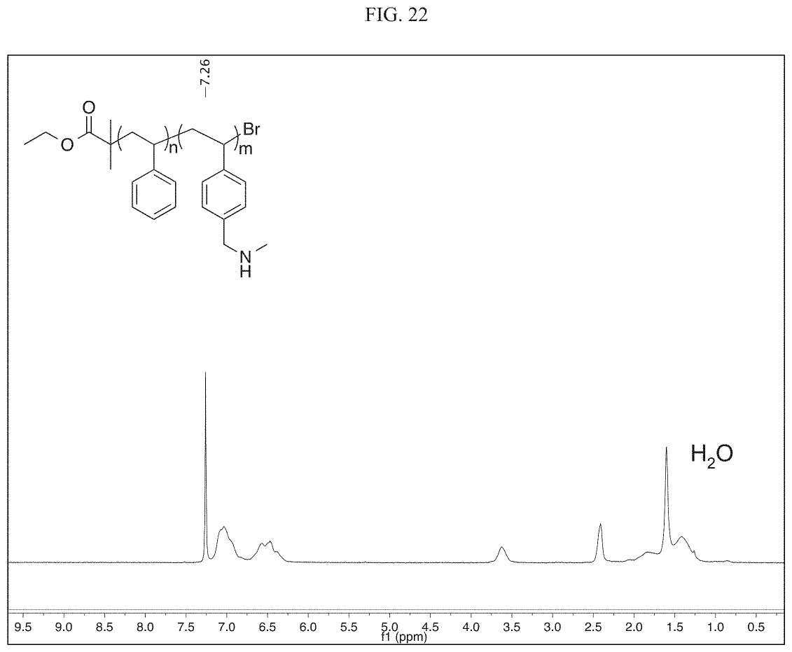

FIG. 23 shows ¹H NMR spectrum of PS-b-PTACCy.

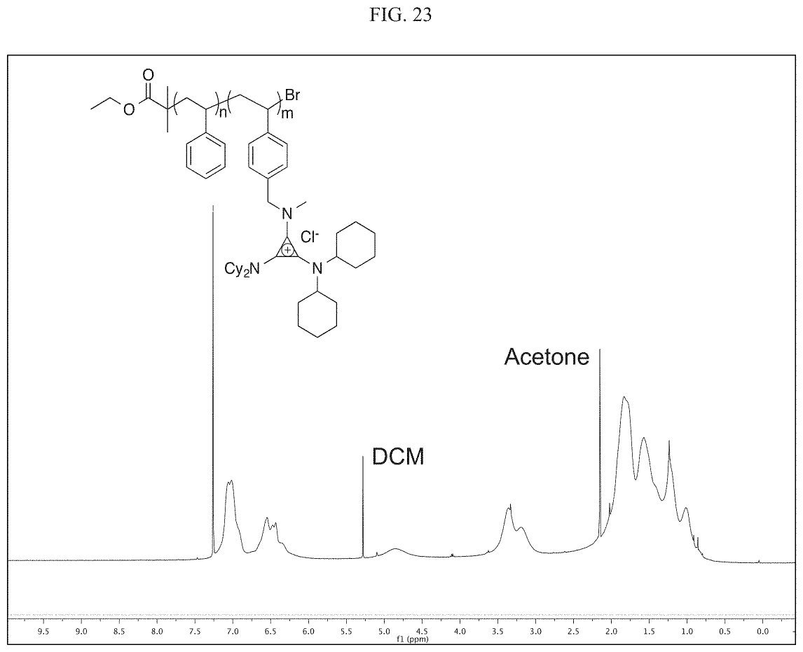

FIG. 24 shows ¹H NMR spectrum of PS-b-PTACiP.

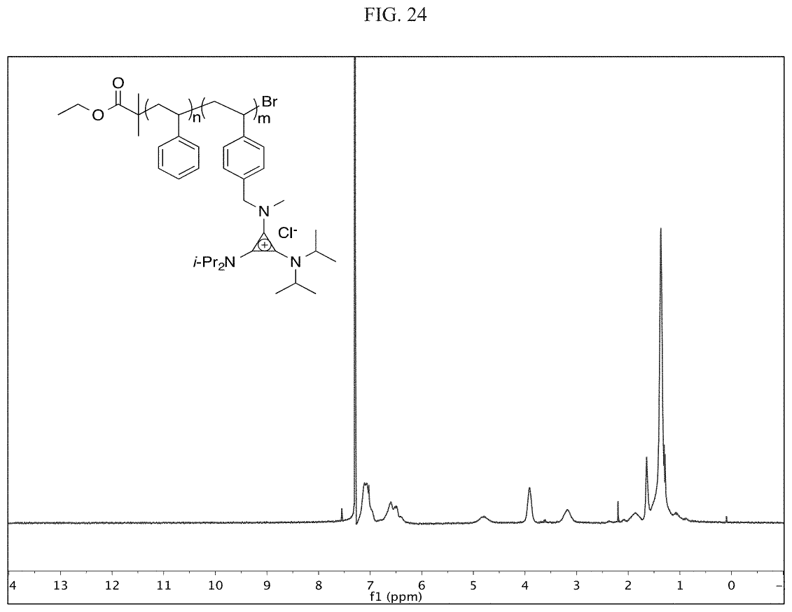

FIG. 25 shows ¹H NMR spectrum of PS-b-PTACMo.

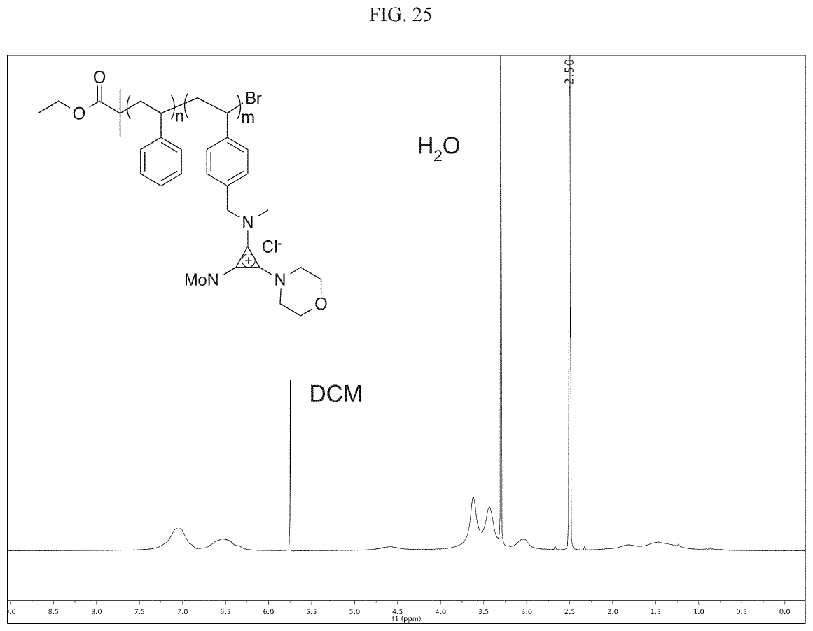

FIG. 26 shows ¹H NMR spectrum of PEO-b-PBoc.

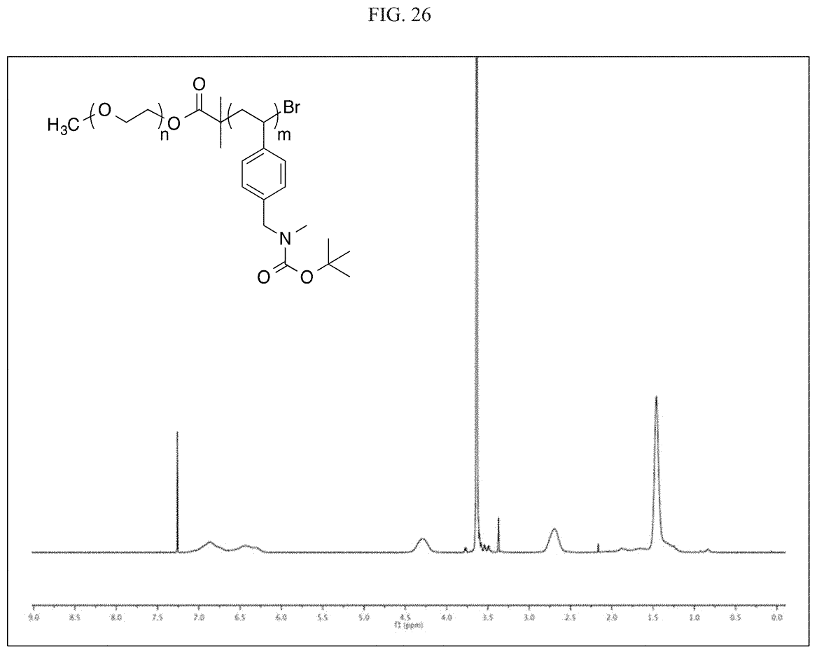

FIG. 27 shows ¹H NMR spectrum of PEO-b-PMAS.

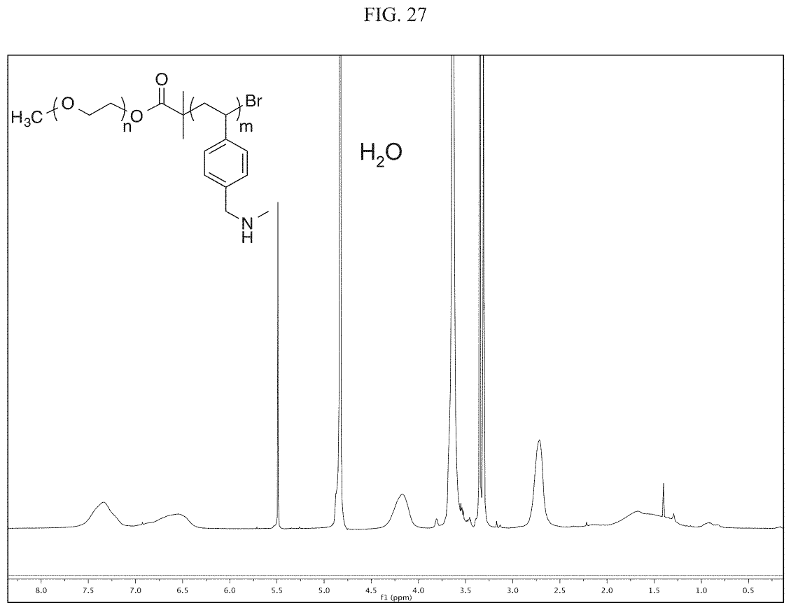

FIG. 28 shows ¹H NMR spectrum of PEO-b-PTACCy.

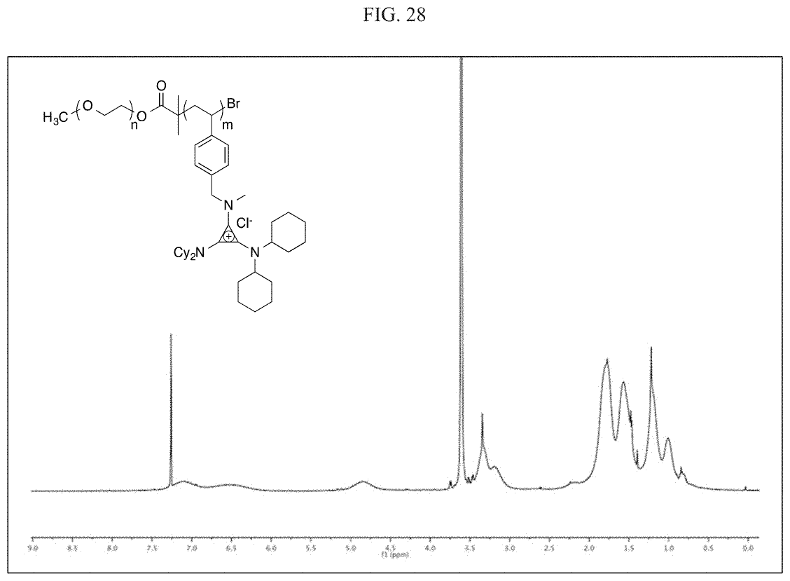

FIG. 29 shows ¹H NMR spectrum of PEO-b-PTACiP.

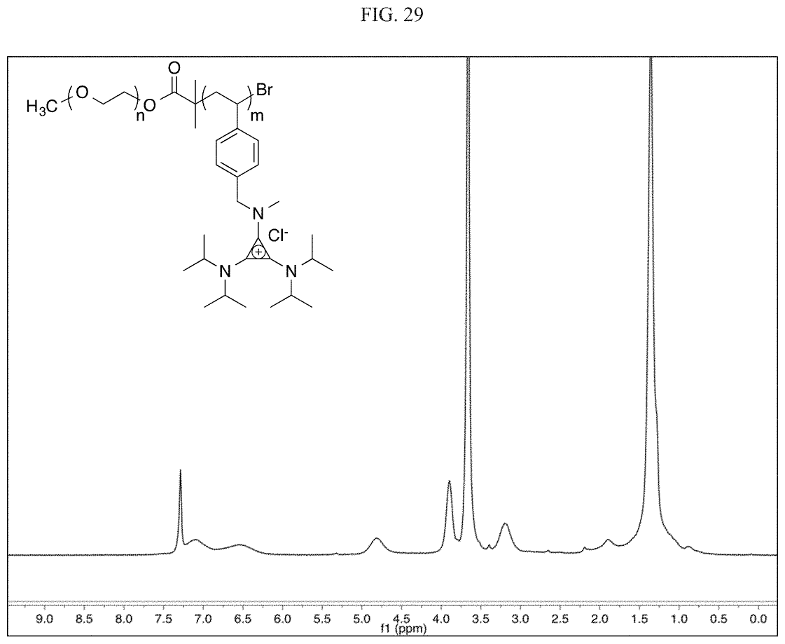

FIG. 30 shows ¹H NMR spectrum of PEO-b-PTACMo.

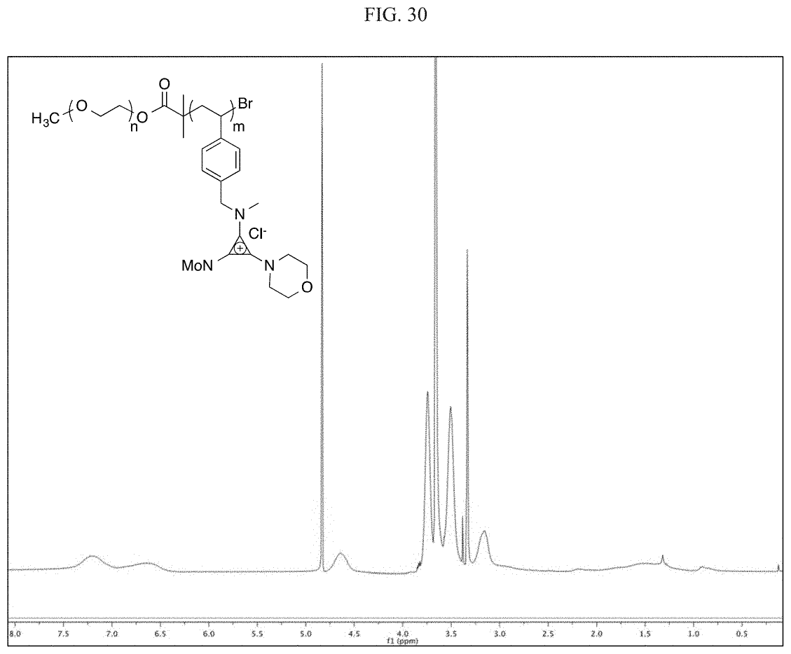

FIG. 31 shows ¹H NMR spectrum of PEI-Cy.

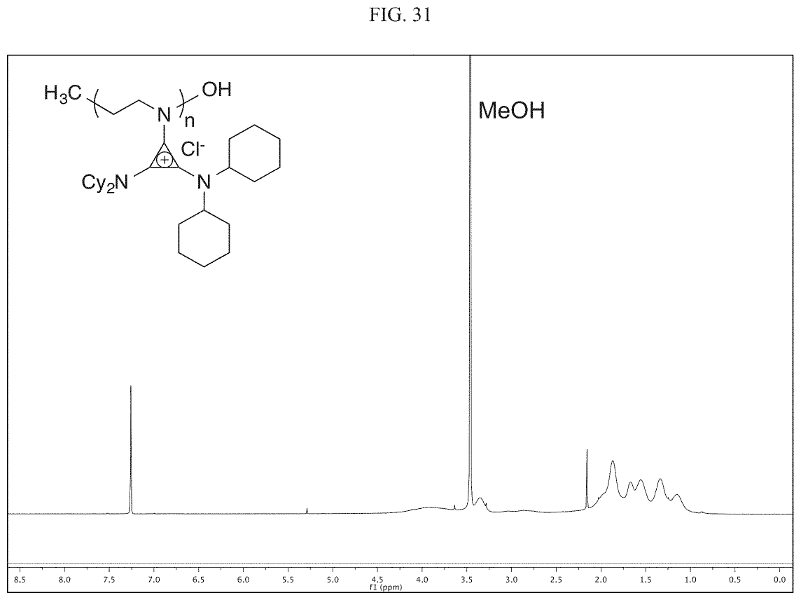

FIG. 32 shows ¹H NMR spectrum of PEI-iP.

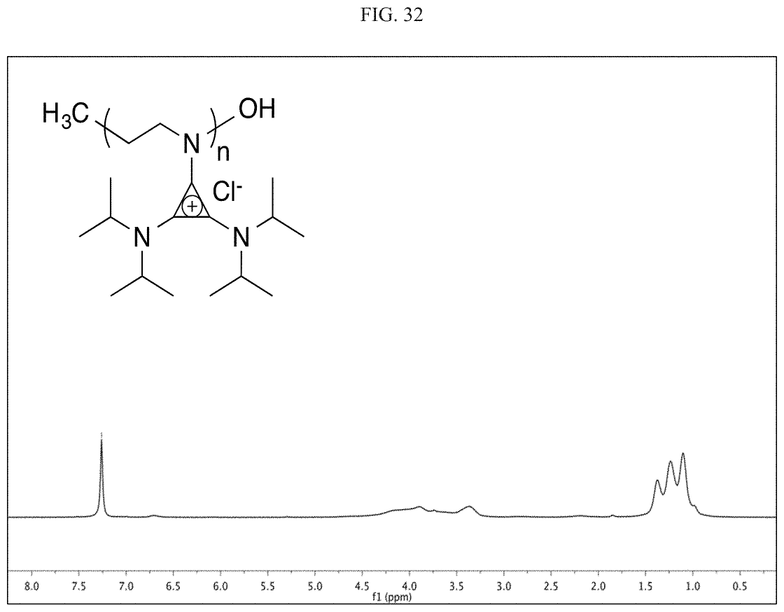

FIG. 33 shows ¹H NMR spectrum of PEI-Mo.

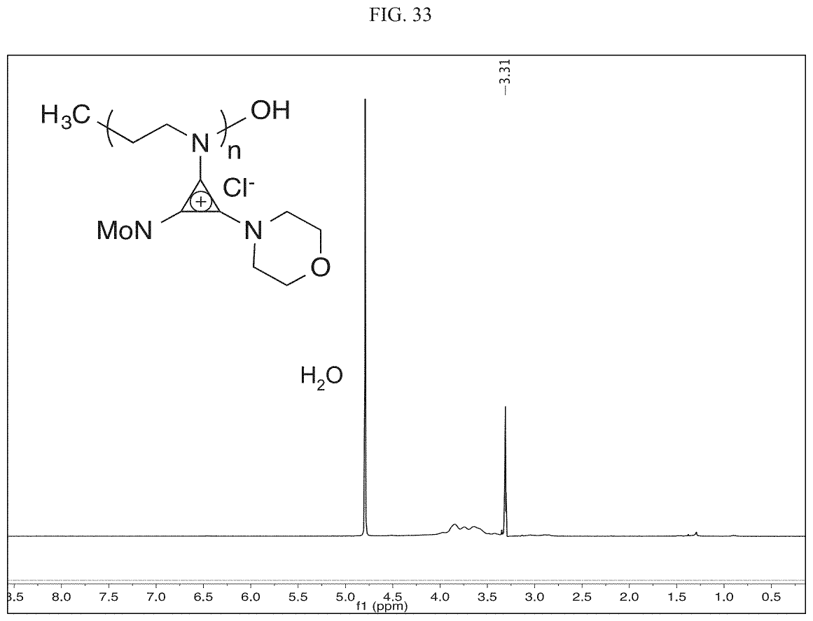

FIG. 34 shows ¹H NMR spectrum of PEI-Pip.

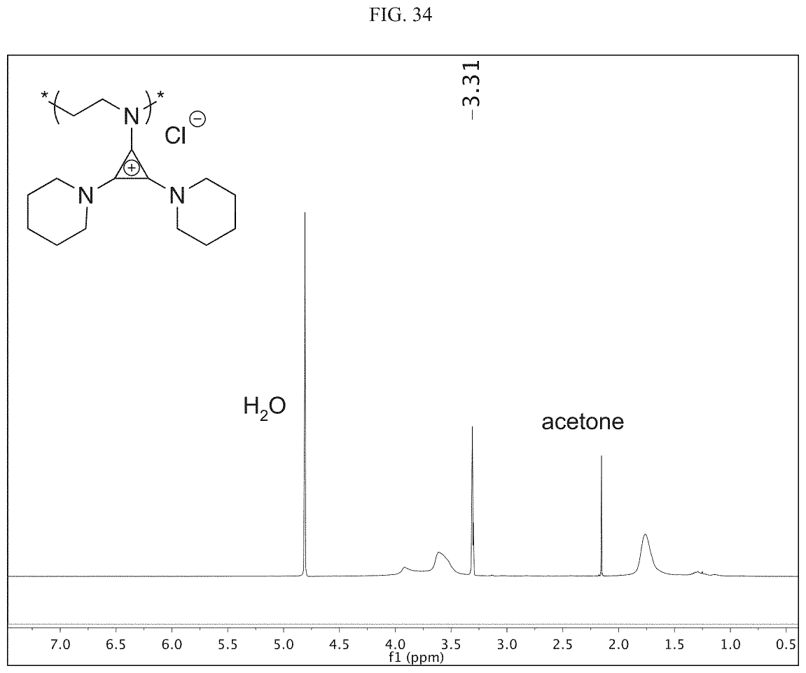

FIG. 35 shows the cell viability of transfected 293T cells as a function of polymer loading for polymers functionalized with BACiP groups. Error bars represent the standard deviation of three measurements.

FIG. 36 shows Luciferase expression of transfected 293T cells with all functionalized polymers across loading series. Error bars represent standard deviation of four measurements.

FIG. 37 shows trisaminocyclopropenium (TAC) polymer structures examined for biocompatibility and transfection efficacy. PEI: polyethylenimine; PMAS: poly(methylaminostyrene); Bu; n-butyl; iP; ispropyl.

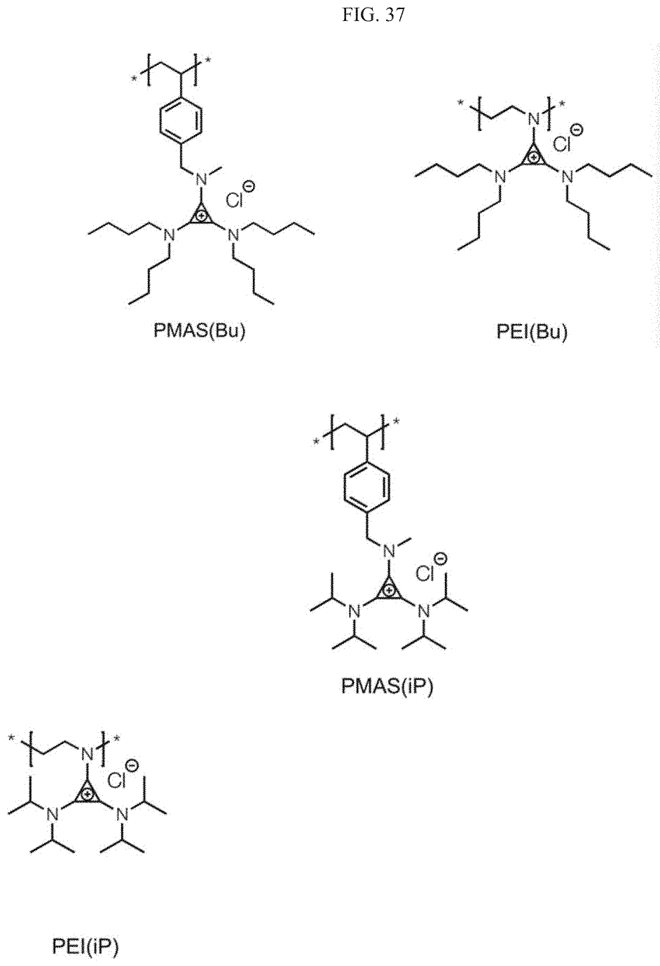

FIG. 38 shows biocompatibility of TAC-based polymers at various doses in HEK-293T cells following 48 h incubation. Viability is measured by trypan blue dye exclusion and normalized to untreated cells. Error bars show the standard deviation of triplicate measurement.

FIG. 39 shows luciferase expression in HEK-293T cells transfected with pDNA containing the firefly luciferase reporter gene using TAC polymers. Luciferase expression is measured after 48 h incubation with specified polymer loadings (all with pDNA loadings of 3 μg·mL⁻¹) and normalized by cell count. Error bars show the standard deviation of triplicate measurement.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

Reference will be made in detail to certain aspects and exemplary embodiments of the present disclosure, illustrating examples in the accompanying structures and figures. The aspects of the present disclosure will be described in conjunction with the exemplary embodiments, including methods, materials and examples, such description is non-limiting and the scope of the present disclosure is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the present disclosure are not limited to the methods and materials described.

Exemplary aspects of the present disclosure relate to applications of cationic poly(ionic liquids) in medicine and scientific research, and the development of efficient synthetic strategies to target innovative cationic building blocks is a consequential goal.

The term “about” as used here refers to the usual error range for the respective value readily known to the skilled person in this technical field. Reference to “about” a value or parameter here includes (and describes) embodiments that are directed to that value or parameter per se.

As used here and in the appended claims, the singular forms “a,” “an,” and “the” include plural reference unless the context clearly indicates otherwise. For example, reference to a “cyclopropenium ion” is a reference to one to many cyclopropenium ions, such as molar amounts, and includes equivalents thereof known to those skilled in the art, and so forth.

As used herein, the term “cyclopropenium ion” can mean a charged species derived from a cyclopropene having the structure:

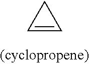

### Method of Synthesis of Cyclopropenium-Ion Containing Polymers for Use as Delivery Agents

An exemplary aspect of the present disclosure relates to the facile synthesis of a series of polymers incorporating cyclopropenium (“CP”) building blocks with various functional groups that acutely affect physical properties. The synthetic routes described and used here to obtain cyclopropenium ion-containing monomers are robust and scalable, and these monomers are easily polymerized by reversible addition-fragmentation chain transfer polymerization. The synthesis or method includes a modular polymeric precursor that allows access to CP macromolecules via a post polymerization strategy with efficiency levels approaching those attained by click chemistry. Macromolecular assemblies of these materials can be used as ion-conducting membranes that offer mechanical integrity and well-defined conducting paths for ionic transport. Moreover, the electric double layer capacitance of CP polyelectrolytes bearing various counter ions was investigated by electrochemical impedance spectroscopy.

### Cyclopropenium Ion Scaffold

The cyclopropenium (CP) ion scaffold addresses the challenges of the limited selection of monomers for developing cationic polyelectrolytes, while offering distinct structural architecture and electronic properties from cationic liquids. Exemplary embodiments of the present disclosure include CP ions as described in U.S. application Ser. No. 14/611,166, which is incorporated by reference herein in its entirety.

An exemplary embodiment of the present disclosure includes where the functionalized cyclopropenium ion is a compound of formula (100):

where

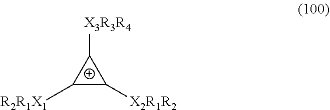

X₁₋₃ are independently selected from the group consisting of Cl, N and any other atoms suitable for participating in the process; and
R₁₋₄ are independently selected from the group consisting of no atom, amino, aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀ cycloalkenyl, C₂₋₁₀alkynyl, halogen, aryloxy, heteroaryloxy, C₂₋₁₀alkoxycarbonyl, C₁₋₁₀ alkylthio, C₂₋₁₀alkenylthio, C₂₋₁₀alkynylthio, C₁₋₁₀alkylsulfonyl, aryl-C₁₋₁₀ alkyl, heteroaryl-C₁₋₁₀alkyl, aryl-C₁₋₁₀heteroalkyl, heteroaryl-C₁₋₁₀heteroalkyl, a phosphorus group, a silicon group and a boron group, wherein R₁ and R₂ or R₃ and R₄ are optionally combined to form a 5 to 8-membered carbocyclic or heterocyclic ring; further wherein the aliphatic or aromatic portions of R₁ and R₂ are optionally substituted with from 1 to 4 substituents selected from the group consisting of halogen, cyano, nitro, C₁₋₄alkyl, C₂₋₆ alkenyl, C₂₋₆alkynyl, aryl, C₁₋₆alkoxy, C₂₋₆alkenyloxy, C₂₋₆alkynyloxy, aryloxy, C₂₋₆ alkoxycarbonyl, C₁₋₆alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus.

Another exemplary embodiment of the present disclosure includes a stable cyclopropenium cation that remains positively charged at a high pH, the polymer having the structure:

where

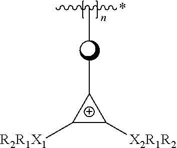

X₁₋₂ are independently selected from the group consisting of or comprising Cl, N, and any other atom suitable for participating in the process;
R₁₋₂ are independently selected from the group consisting of no atom, amino, aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀ cycloalkenyl, C₂₋₁₀alkynyl, halogen, aryloxy, heteroaryloxy, C₂₋₁₀alkoxycarbonyl, C₁₋₁₀ alkylthio, C₂₋₁₀alkenylthio, C₂₋₁₀alkynylthio, C₁₋₁₀alkylsulfonyl, aryl-C₁₋₁₀ alkyl, heteroaryl-C₁₋₁₀alkyl, aryl-C₁₋₁₀heteroalkyl, heteroaryl-C₁₋₁₀heteroalkyl, a phosphorus group, a silicon group and a boron group, wherein R₁ and R₂ are optionally combined to form a 5 to 8-membered carbocyclic or heterocyclic ring; further wherein the aliphatic or aromatic portions of R₁ and R₂ are optionally substituted with from 1 to 4 substituents selected from the group consisting of halogen, cyano, nitro, C₁₋₄alkyl, C₂₋₆alkenyl, C₂₋₆alkynyl, aryl, C₁₋₆ alkoxy, C₂₋₆alkenyloxy, C₂₋₆alkynyloxy, aryloxy, C₂₋₆alkoxycarbonyl, C₁₋₆alkylthio, C₁₋₆ alkylsulfonyl, C₁₋₆alkylsulfinyl, oxo, imino, thiono, primary amino, carboxyl, C₁₋₆ alkylamino, C₁₋₆dialkylamino, amido, nitrogen heterocycles, hydroxy, thiol and phosphorus;

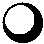

represents a suitable linking group; and
n is an integer.

### Polymer Backbone Precursors

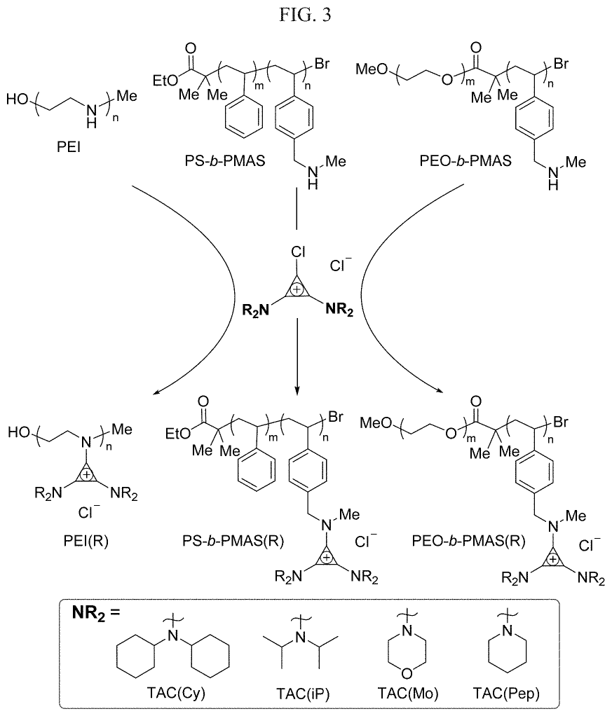

The polyamines used in the present application include linear polyethyleneimine and polyvinylbenzylmethylamine (see, e.g., Example 7 and FIG. 3). Polyamines may include but are not limited to polyamines comprising a polymer containing a secondary amine, poly(amine-co-esters), PEI, PS-b-PMAS, PEO-b-PMAS, and polyvinylbenzylmethylamine.

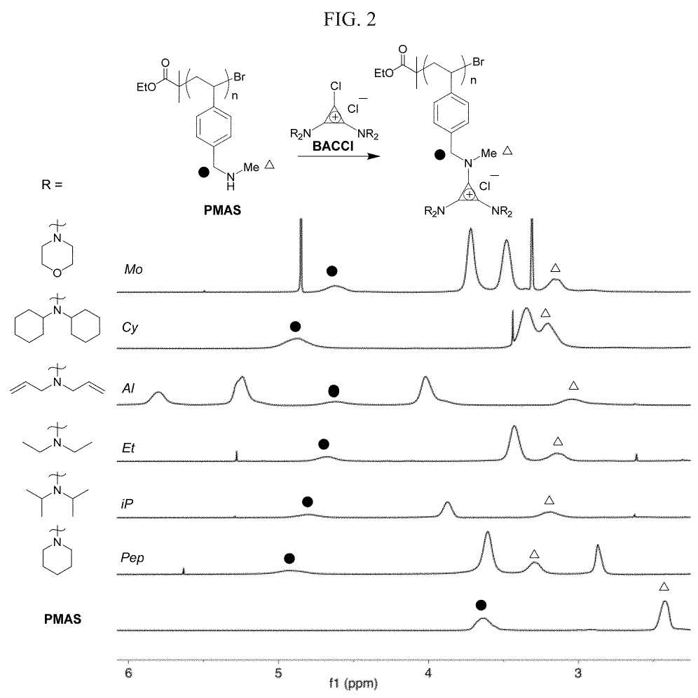

One exemplary embodiment is directed to a well-defined neutral polymer precursor, poly(methylaminostyrene) (PMAS, FIG. 2), a polymer containing a secondary amine as a pendant group. Because monomeric PMAS does not polymerize by RAFT or atom-transfer radical-polymerization (ATRP) conditions, the secondary amine was protected with a tert-butyloxycarbonyl (Boc) protecting group. [Ref. 24] The Boc-protected monomer readily polymerizes by ATRP to yield polymers of controllable molecular mass and narrow dispersity (D), which can be characterized by size exclusion chromatography at this step. Further details of the synthetic protocols are available in the Examples section.

### Post-Polymerization Functionalization

The post polymerization chemistry towards cyclopropenium-containing polymers enables precise structural tuning to optimize delivery agent structure, including cellular transfection agent structure. Essentially the post polymerization chemistry provides acute control over molecular structure to enable optimization of the delivery agents based on cyclopropenium and serves as a way to systematically study how structural variations within a family of materials impacts, for example, delivery or transfection efficacy. Furthermore, these cyclopropenium polymers coupled with the post polymerization functionalization chemistry can be used as tools to discovery of design principles for polymeric transfection agents, etc.

More specifically, e.g., a post-polymerization click reaction that provides facile access to trisaminocyclopropenium (TAC) ion-functionalized macromolecules of various architectures, which are the first class of polyelectrolytes bearing a formal charge on carbon, is described here. Quantitative conversions of polymers comprising pendant or main-chain secondary amines were observed for an array of TAC derivatives in about three hours using near equimolar quantities of cyclopropenium chlorides. Therefore, these reactions are designated as “click” in the context of polymer chemistry, [Ref. 23] establishing the bis(dialkylamino)cyclopropenium chloride (BACC1) ion as a clickable ionic liquid, or ClickabIL. One exemplary embodiment of the present disclosure is directed to a new type of click reaction between BACC1 ILs [Ref. 21] and polymers containing secondary amines, along with a demonstrative transfection study. TAC polymers described here are biocompatible and efficient transfection agents, and that ClickabIL chemistry allows for straightforward screening of polymeric TAC derivatives. This robust, efficient, and orthogonal click reaction provides a modular route to synthesize and study various properties of novel CP-based polymers, including but not limited to delivery systems, such as, for example, their propensity to serve as cellular transfection agents.

While many literature procedures to obtain other cationic PILs via PPF have reported reactions with large excesses of the quaternizing agent (3-10 eq) and long reaction times (up to three days), [see Ref 22] the conjugation reaction described here proceeds in, at, or about 3 hours under mild conditions, with stoichiometric amounts of reactants. The smallest Huckel aromatic cycle can efficiently react in the form of a stable BACC1 salt with polymers containing secondary amines to yield cationic polyelectrolytes. By modulating the dialkylamino groups, the click transformation yields a library of a new class of polyelectrolytes bearing soft cationic moieties. The dialkylamino groups used in the described embodiments include, but are not limited to, dicyclohexylamine, diisopropylamine, morpholine, piperidine, diallylamine, and diethylamine.

A variety of BACC1 building blocks readily react with polymers containing pendant and main-chain secondary amines in a novel example of macromolecular click chemistry. Linking neutral polyamines that are either commercially available or rapidly assembled, with charged BACC1 ClickabILs furnishes diverse classes of well-defined TAC polymers under mild conditions.

The deprotected PMAS undergoes smooth addition to BACC1 salts, as shown in FIG. 2. The choice of BACC1 can tailor the physical properties of the resulting polymers through control of solubility properties or through the introduction of functional groups via the amino substituents (e.g., dialkylamino). The synthesis of these ClickabILs can be accomplished in high yields from inexpensive and readily available materials, in one-to-three steps (see, Examples). PMAS was subjected to functionalization with six different BACC1 derivatives containing isopropyl (iPr), ethyl (Et), allyl (Al), cyclohexyl (Cy), morpholine (Mo), and piperidine (Pip or Pep) substituents (FIG. 2). Proton nuclear magnetic resonance (¹H NMR) spectra of these polymers reveal that the starting material is fully converted into the corresponding TAC-containing polymer, as evidenced in the peak shifts noted in FIG. 2. Notably, the positive charge is not generated by a reaction between neutral reactants in this new approach, in contrast to quaternization PPF reactions.[Ref. 25] Instead, BACC1 salts are directly coupled to neutral homopolymers containing secondary amines. While there may be some differences in the solubility profiles and thermal properties of TAC polymers comprising different amino substituents, [Ref. 9a] this chemistry provides a direct approach to study the impact that various functional groups exert on macromolecular properties and increases access to diverse forms of polymeric TACs.

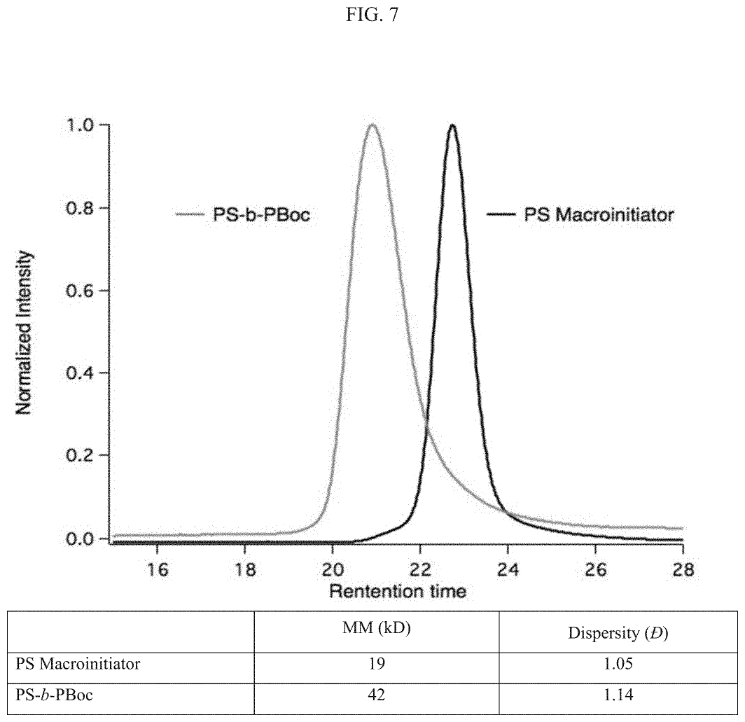

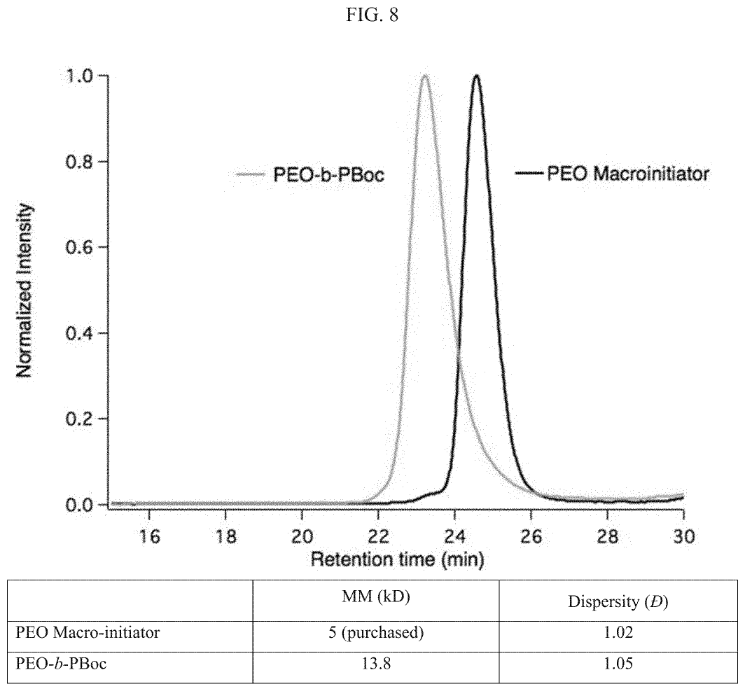

The modularity of this protocol is highlighted by the diverse set of functional groups obtained using the same parent polymer; only minor changes in procedure (e.g. use of co-solvent) are needed to accommodate structural diversity. In this vein, diblock copolymers were synthesized by polymerization of the Boc-protected monomer onto both polyethylene oxide and polystyrene macro-initiators by ATRP (FIG. 3). SEC traces show a narrow dispersity is maintained after copolymerization in both cases (FIG. 7 and FIG. 8). Following their successful deprotection, the PMAS blocks in PEO and PS diblock copolymers were fully functionalized with both hydrophobic and hydrophilic BACC1s (Cy, iPr, and Mo, from most to least hydrophobic) without the need to modify the procedures used to prepare corresponding homopolymers. The commutable nature of this chemistry to obtain materials of different physical properties within the same family of polymers will encourage systems development away from individual polymer design.

In order to expand upon the macromolecular architectures accessible with ClickabIL chemistry, commercially available linear polyethyleneimine (PEI) was functionalized with bis(dialkylamino)cyclopropenium chlorides (BACC1s). When protonated, PEI is a cationic polyelectrolyte frequently used as a cellular transfecting agent. [See Ref. 26] However, its cytotoxicity, especially at high doses, limits its widespread use in transfection applications.[Ref. 27] It was postulated that functionalizing PEI with cyclopropenium units might make it less toxic, and potentially, a better transfection agent. PEI was subjected to the ClickabIL reaction conditions described above (FIG. 3), and quantitative functionalization was observed for all BACC1s.

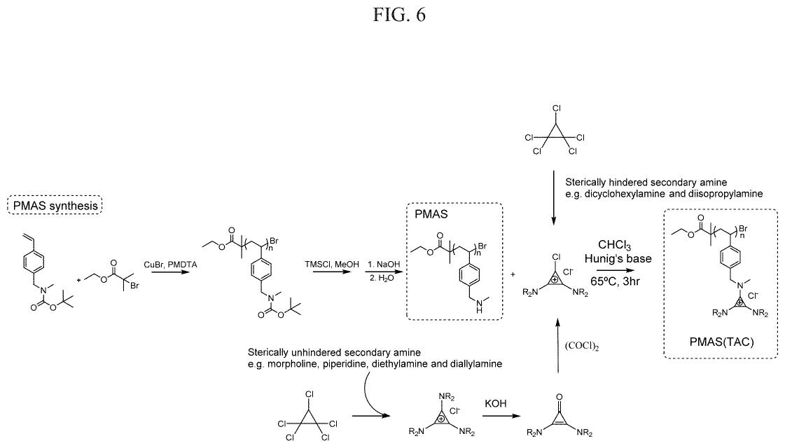

FIG. 6 shows an exemplary scheme for the synthesis of PMAS(TAC). PMAS is initially synthesized. A sterically unhindered secondary amine, such as but not limited to, morpholine, piperidine, diethylamine, and diallylamine, is reacted with 1,1,2,2,3-pentachlorocyclopropane, further reacted with potassium hydroxide (KOH) and then reacted with oxalyl dichloride to form, in addition to another 1,1,2,2,3-pentachlorocyclopropane that is reacted with a sterically hindered secondary amine, such as for example, dicyclohexylamine and diisopropylamine, a TAC product that is then reacted with PMAS to form PMAS(TAC).

### Cyclopropenium Ion-Containing Polymers Significantly Lower Cytotoxicity of Highly Charged Non-Ionic Delivery Agents

Select polymers were analyzed for their viability as gene delivery agents, focusing on chemical structure variations of the materials. Some cyclopropenium-based polymers have high transfection efficiencies and are less cytotoxic than linear polyethyleneimine alone which is presently a widely used commercially-available cationic polymer transfection agent. Biocompatibility and transfection capacity were highly dependent on BACC1 identity and polymer backbone, and some materials showed similar transfection efficiencies to PEI with lower cytotoxicities. This chemistry may be further used to explore a divergent approach to materials discovery and optimization of cyclopropenium macromolecules for a variety of applications.

These polymers containing derivatives of cyclopropenium ions synthesized by the PPF strategy have advantages over the closest technology in the art. For example, the described cyclopropenium-based polymers embodied in the application have a no pH dependent charge within the range of about pH 2-12, and the formal charge lies on carbon atoms rather than on heteroatoms, such as for example, Nitrogen or Phosphorous. In extreme pH conditions, such as for example pH 14, the charge is not stable. Exemplary embodiments of the present application are directed to derivatized cyclopropenium (CP) ion-containing polymers or complexes, their syntheses, and their use as delivery agents, for example, for drug delivery or as cellular transfection agents for gene delivery. These CP-containing polymers unexpectedly include those that lack significant cytotoxicity at loadings where transfection was accomplished and demonstrate good transfection efficacy.

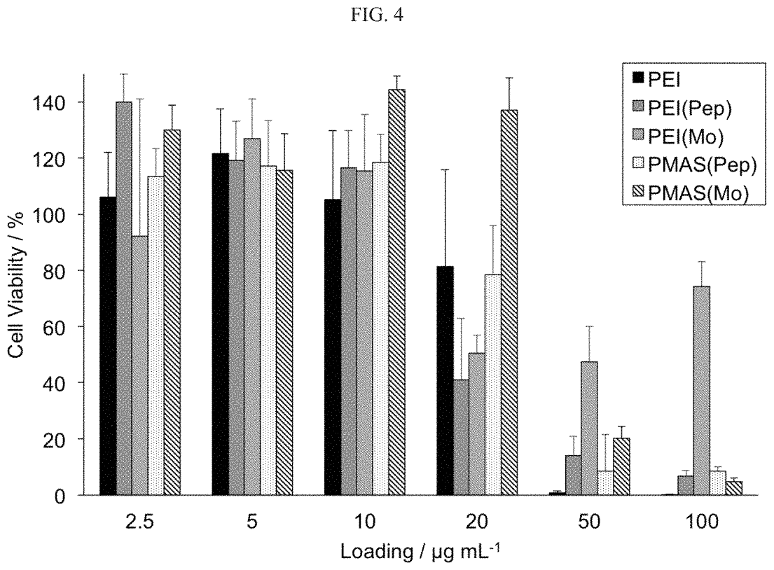

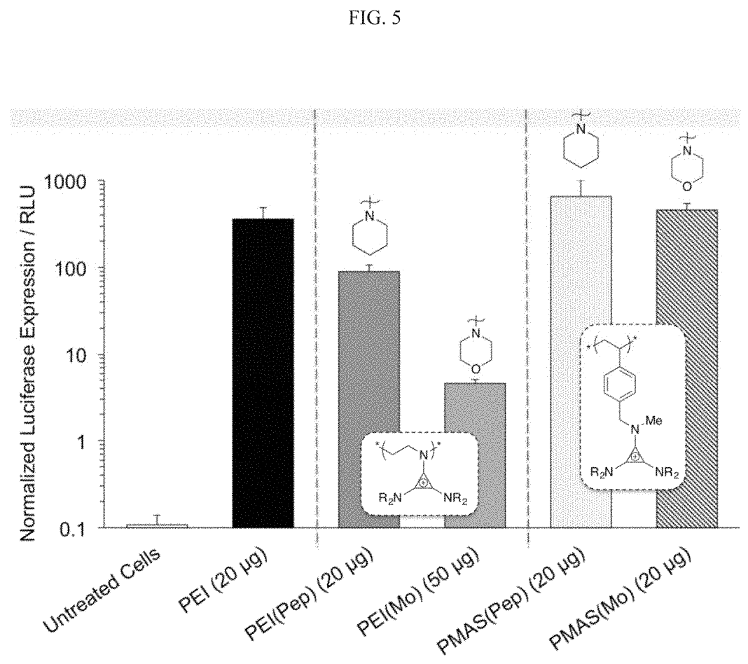

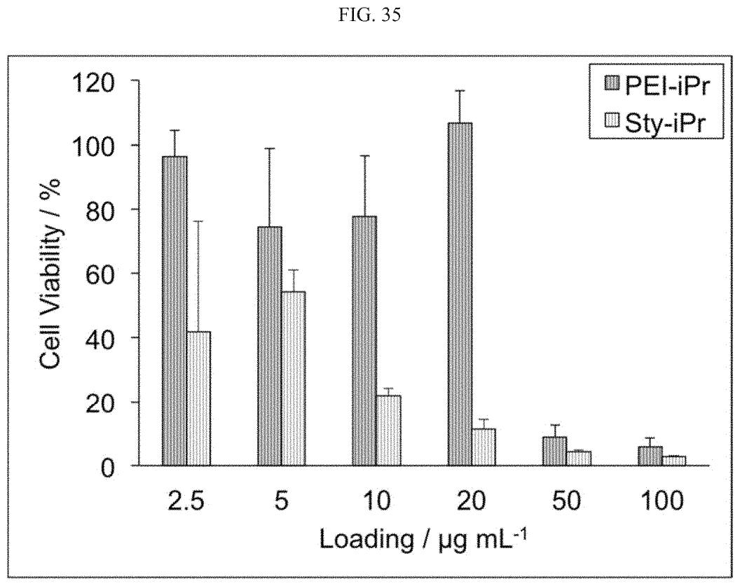

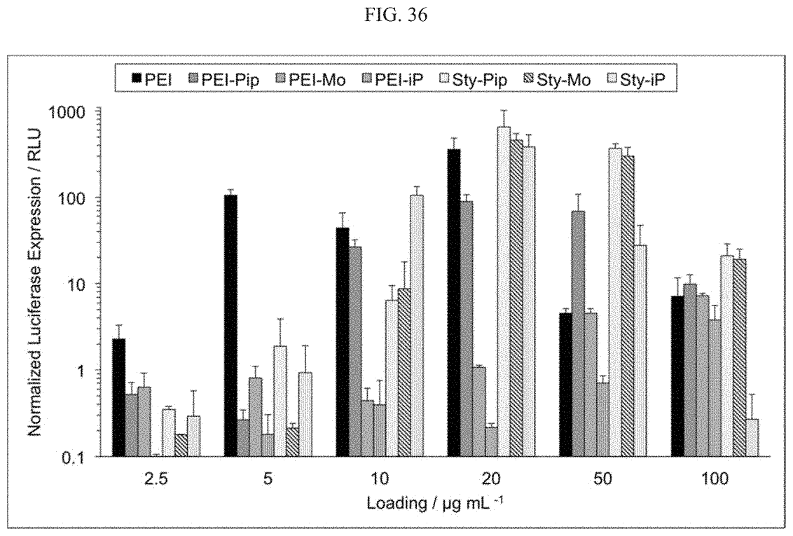

For biomedical applications, such as gene delivery, cytotoxicity has been a limiting factor. Thus, one exemplary embodiment of the present disclosure is directed to TAC-based polymers that are safe and non-cytotoxic. Cytotoxicity assays and luciferase transfections in HEK-293T cells revealed a significant dependence on the chemical structure of the pendant TAC ion, namely its amino substituents, and on the identity of polymer backbones: PEI and PMAS. All four TAC polymers showed a similar toxicity profile to linear PEI (25 kg mol⁻¹) at low dosages. However, at high loadings, where PEI is highly cytotoxic, polyTACs were found to be more biocompatible (PEI-Pip, PEI-Mo, PS-Pip, and PS-Mo), especially those bearing a styrene backbone (FIG. 4, PS-Pip, and PS-Mo). Functionalizing PEI with TACMo endowed the polymer with a cell viability of ˜50% at both 50 and 100 μg mL⁻¹ loadings, representing a dramatic improvement from unfunctionalized PEI. See, e.g., FIG. 5, FIG. 35, and FIG. 36.

While the structural modification of PEI with the TAC ions led to a lower transfection efficacy as compared to the parent polymer (see FIG. 5), comparing the two modified PEI materials bearing TACPip and TACMo, led to notable differences in transfection efficiency. These two materials differ in the chemistry at the 4-position of the six-membered ring—a variation between a methylene group and an ether oxygen. Here, PEI-Pip polyplexes transfected cells almost as well as the PEI parent polymer, but polyplexes of PEI-Mo exhibited poor transfection efficiency (see FIG. 5). This difference may be attributed to the increased hydrophobic nature of PEI-Pip over PEI-Mo, which has been shown to play a key role in non-viral transfection agents. [See Ref. 28] Polyplexes of PEI and TAC polymers with pDNA, at the loadings noted in FIG. 5, were further characterized by dynamic light scattering for their hydrodynamic diameter (DH) and zeta (0 potential (Table 1).

Table 1 shows the characterization of transfection agents and polyplexes corresponding to optimal transfection efficacy.

TABLE 1

| Transfection | MM^{[b]} | Charge | DH | ζ potential |
|---|---|---|---|---|
| Agent^{[a]} | [kDa] | ratio^{[c]} | [nm] | [mV] |
| PEI | 25 | 50:1 | 490 ± 60 | 40 ± 10 |
| PEI(Pep) | 166 | 8:1 | 425 ± 100 | 65 ± 5 |
| PEI(Mo) | 164 | 20:1 | 400 ± 110 | 60 ± 6 |
| PMAS(Pep) | 53 | 5.5:1 | 140 ± 60 | 27 ± 8 |
| PMAS(Mo) | 53 | 5.5:1 | 215 ± 25 | 43 ± 6 |

[a] Polyplexes of polymers tested at loadings noted in FIG. 5. [b] Molecular mass of transfection agent, calculated based on commercial linear PEI; for PS materials, PMAS was measured by SEC using PS standards of narrow dispersity, then calculated for corresponding TAC group. [c] Ratio of either N to phosphate anion (PEI) or TAC to phosphate anion.

As the hydrodynamic diameter (DH) and surface charge of PEI-Pip, PEIMo, and unfunctionalized PEI polyplexes are similar (Table 1), the observed discrepancy in transfection efficacy may be due to fine structural variations between each agent. The complex relationships between transfection efficiency and TAC structure, polymer molecular mass, and Coulombic interactions are undergoing further investigation. [See Ref 29] Changing the backbone from PEI to polystyrene (PS) resulted in smaller polyplexes and afforded transfection agents that were both less cytotoxic and more efficacious in transfection than PEI (FIG. 5, PS-Pip and PS-Mo). All of the most effective formulations (Table 1 and FIG. 5) are within the size regime that Zhou and coworkers outlined for highest efficiency transfection reagents, i.e. sub 500 nm. [See Ref. 29] Furthermore, PS-based materials exhibited optimal pDNA transfection at lower charge ratios than PEI and PEI-TAC polymers (Table 1). Within the range of the error bars, both the more hydrophobic piperidine and the morpholine PS derivatives are similar, unlike in the PEI systems. These results suggest ClickabIL chemistry as a platform to both tune the chemical composition of polyelectrolytes and build detailed structure-property relationships.

Yet a further exemplary embodiment of the present disclosure includes various polymers containing cyclopropenium ion derivatives of interest as delivery agents, such as for example, cellular transfection agents for gene delivery. These cyclopropenium-containing polymers may complex with plasmid DNA and act as transfection reagents when incubated with cells by delivering the nucleic acids to cells. The polymers are beneficial as they do not display significant cytotoxicity at loadings where transfection was accomplished and do display transfection efficacy. Polymers containing the cyclopropenium derivatives include the following:

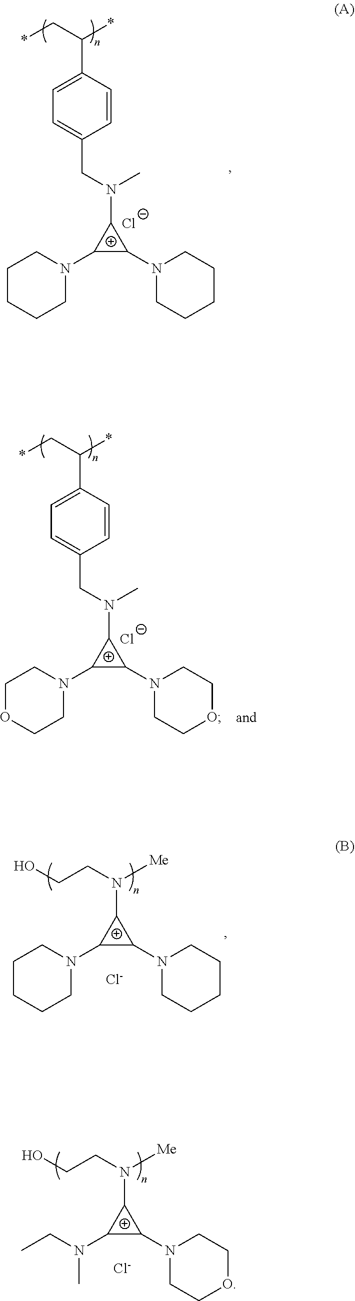

The examples of (A) polymers may have n=100-200, preferably n=130-140, and more preferably n=135, where the polymer may be, but not limited to, PMAS(Pep) and PMAS(Mo):

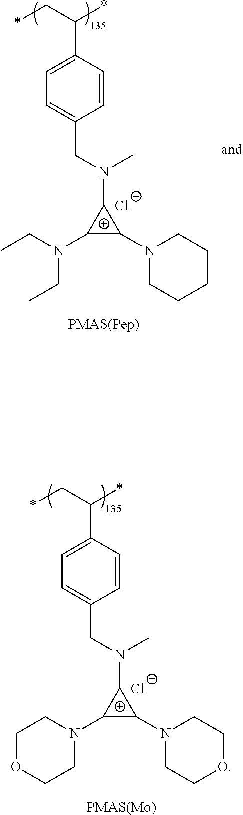

The (B) polymers may have n=220-590, preferably n=575-585, and more preferably n=581, where the polymer may be, but is not limited to, PEI(Pep) and PEI(Mo):

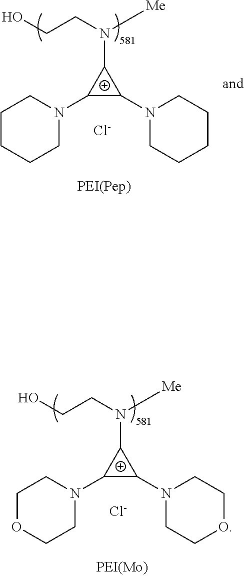

Further analysis focused on two BACC1 structures (BAC(Bu) and BAC(iP)) comprising dialkylamino substituents differing in the degree of branching, and thereby “floppiness”, as well as hydrophobicity, as the Bu-derivatized polymers have one more carbon. Complete functionalization of both BAC(Bu), containing n-butyl substituents, and BAC(iP), containing isopropyl substituents, was confirmed by proton nuclear magnetic resonance spectroscopy. Quantitatively functionalizing polymers holds effects of dispersity and degree of polymerization constant, permitting direct comparisons of subtle structural changes on macromolecular properties.

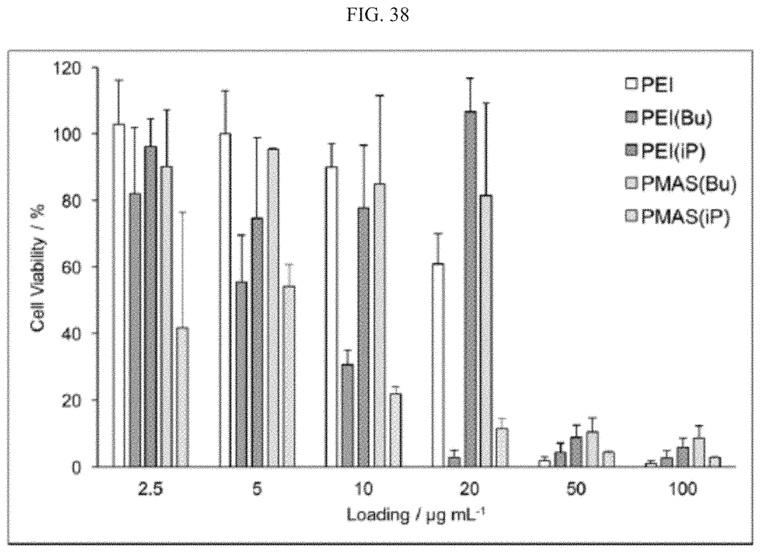

To probe the impact of alkyl chain conformation on cell viability and transfection efficiency, the series of TAC-functionalized polymers were assessed and compared as vectors via cytotoxicity assays and luciferase transfection experiments in HEK-293T cells. All four homopolymers (PMAS(Bu); PEI(Bu); PMAS(iP); PEI(iP)) were highly water-soluble, permitting their condensation with an aqueous solution of plasmid DNA (pDNA) containing the firefly luciferase reporter gene. Combining the polymers at varied loadings with a fixed amount of pDNA, and subsequently incubating in cells for 2 days, revealed the polymers' biocompatibility as a function of loading. PEI(iP) and PMAS(Bu) were the most biocompatible with high cell viabilities through loadings of 20 μg·mL⁻¹ (FIG. 38).

In order to assess the amount of polymer necessary to completely condense pDNA into a polyplex, gel electrophoresis shift assays were performed. While all polymers were able to fully bind the pDNA by a weight ratio of 3:33:1, PEI(iP) was the most efficient, binding at a weight ratio of only 0.83:1. This corresponds to the lowest polymer loading tested for either biocompatibility or transfection, and less than 1 TAC unit per phosphate anion of pDNA.

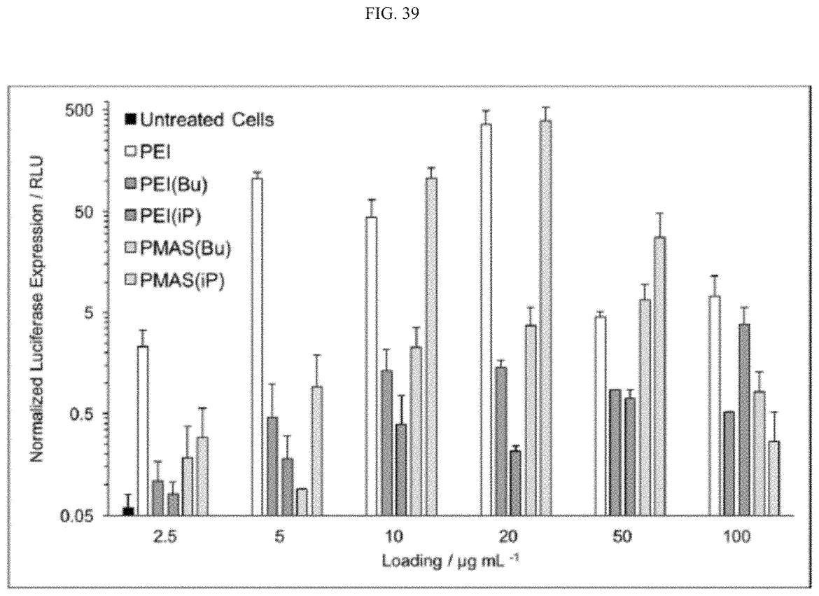

While all TAC-based polymers transfected pDNA significantly better than the untreated controls, PMAS(iP) demonstrated the highest transfection efficacy (FIG. 39). As is the case with unmodified linear PEI, successful delivery of intact pDNA to cells comes at the cost of significant cytotoxicity. By contrast, the nontoxic PEI(iP) demonstrated a much lower luciferase activity. Interestingly, PEI(iP) was the most efficient at compacting pDNA into a polyplex, suggesting that it binds nucleic acids too strongly and never releases its payload. Converting either of the polymer backbones into a TAC bearing n-butyl chain seemed to yield successful nonviral vectors capable of both binding and slowly releasing pDNA. This could potentially be attributed to critical destabilization of the cell and endosomal membranes due to the long, flexible alkyl substituents. At their optimal loadings, both PEI(Bu) and PMAS(Bu) demonstrated two orders-of-magnitude improvement over untreated control cells. Taken together with the cytotoxicity and pDNA-binding data, amongst this family of TAC polymers, PMAS(Bu) is the most preferred nonviral vector.

Dynamic light scattering determined the hydrodynamic diameter (D_{H}) and zeta potential (ζ) of the polyplexes at their optimal loading for transfection efficacy (Table 2). All four cationic polymers formed stable polyplexes of small sizes and highly positive charge. The polyplexes all exhibit a hydrodynamic diameter in the size regime considered optimal for successful gene transfection. Notably, the polymers modified with BAC(iP) resulted in more positively charged polyplexes than those with BAC(Bu) which could be a result of enhanced hydrophobic screening of the charge by the longer, flexible n-butyl chains.

TABLE 2
Characterization of transfection agents and
polyplexes at optimal transfection efficacy.

| Transfection | MM² | Charge | D_{H} | ζ potential |
|---|---|---|---|---|
| Agent¹ | [kDa] | Ratio³ | [nm] | [mV] |
| PEI(Bu) | 215 | 6:1 | 110 ± 40 | 45 ± 7 |
| PEI(iP) | 182 | 35:1 | 100 ± 40 | 58 ± 5 |
| PMAS(Bu) | 25 | 12:1 | 150 ± 50 | 31 ± 8 |
| PMAS(iP) | 21 | 5:1 | 160 ± 40 | 44 ± 6 |

¹Polyplexes of polymers at the loading corresponding to highest transfection efficacy in FIG. 39.
²Molecular mass of transfection agent, calculated based on commercial linear 25k PEI; for PMAS(R) materials, PMAS was measured by gel permeation chromatography (GPC) calibrated using polystyrene (PS) standards of narrow dispersity, then calculated for the corresponding TAC group.
³Ratio of TAC to phosphate anions.

### Use of Cyclopropenium Ion-Containing Polymer Polyplexes as Delivery Agents for Gene Therapy

Such cyclopropenium-based polymers or trisaminocyclopropenium (TAC) ion-functionalized macromolecules may be used and applied to a broad range of fields, including but not limited to, energy storage, electronic devices, medicine, delivery systems, and the like. A preferred use is in the field of medicine, where these TAC based polymers having a formal charge on carbon, may facilitate non-viral gene delivery. These cyclopropenium-containing polymers can complex with plasmid DNA (pDNA) and act as non-viral transfection agents or reagents when incubated with cells by delivering the nucleic acids to the cells. One of the advantages of these polymers is that they unexpectedly do not display significant cytotoxicity at loadings upon successful transfection. Exemplary embodiments of the present application generally relate to CP-based advanced materials that can effectively deliver molecules, such as for example, genetic material to cells, and the chemistry to synthesize such materials enables a rapid method and ultimately results in a non-cytotoxic and efficient delivery system.

These polymers may complex to biologically-active materials, for example, non-limiting examples of proteins, small molecules, or genetic material such as for example, a DNA molecule. The complex or polyplex of the delivery agent and the genetic material may be transfected into cells. These cells may be those from a mammal or a plant. Mammals include, but are not limited to domesticated animals (e.g., cows, sheep, cats, dogs, and horses), primates (e.g., humans and non-human primates such as monkeys), rabbits, and rodents (e.g., mice and rats). In certain embodiments, an individual, a subject, or a patient who is in need of treatment for a disease, condition, or symptom may be treated by transfecting a drug, a genetic material (e.g., DNA), or the like that is complexed to a TAC ion-functionalized macromolecule or TAC-based polymer non-viral delivery agent into one or more cells of the individual, the subject, or the patient.

As used herein, a “gene therapeutic vector” or “gene delivery agent” can mean a vehicle used to transfer genetic material to a target cell. The gene therapeutic vector may be administered in vivo or in vitro. Preferably the cell is a mammalian cell, but other types of cells, e.g., insect, plant, or fungal, or non-mammalian vertebrate cells may be used. The terms “non-viral transfection agent” or “non-viral delivery agent” may also be used where delivery of a specific genetic material or nucleic acid is not being specifically referenced.

In operation, the genetic material may be nucleic acids, such as DNA, RNA, RNAi, mRNA, tRNA, short hairpin RNA (shRNA), short interfering RNA (siRNA), double-stranded RNA (dsRNA), transcriptional gene silencing RNA (ptgsRNA), Piwi-interacting RNA, pri-miRNA, pre-miRNA, micro-RNA (miRNA), or anti-miRNA (as described, e.g., in U.S. patent application Ser. Nos. 11/429,720, 11/384,049, 11/418,870, and 11/429,720 and Published International Application Nos. WO 2005/116250 and WO 2006/126040), and is reversibly linked to one or more of the dendritic core branches of the cyclopropenium-containing polymers. Upon delivery of the gene therapeutic vector to the target cell, the nucleic acid(s) are released.

Imaging of cyclopropenium-containing polymer/pDNA polyplexes in action can be carried out by complexing different polymer types with FITC-labeled plasmid DNA and then obtaining images of the colloidal complexes in HeLa cells over time. Images captured from the confocal laser scanning microscope represent the diffracted light from polymer/pDNA polyplexes; stereology and flow cytometry are also techniques that may be used for visualization.

DNA-macromolecular polyplexes can be delivered by various routes, including intravenous infusion or oral ingestion (e.g. an aerosol via a nebulizer). One method to overcome delivery into the liver has been hydrodynamic transfection. Modifications of the procedure for localized delivery into the hepatic vasculature can be used in larger mammals and humans. Similarly, muscle delivery of naked DNA can be achieved by isolation of the vasculature and pressured delivery into isolated skeletal muscle groups or cardiac muscle by infusion into the coronary arteries and/or direct injection into the myocardium. Other delivery technologies, including electroporation and/or ultrasound-guided DNA uptake, can be used. One of skill will understand the various methods by which a non-viral gene delivery agent can be delivered (Yin, H.; Kanasty, R. L.; Eltoukhy, A. A.; Vegas, A. J.; Dorkin, J. R.; Anderson, D. G. Non-viral vectors for gene-based therapy. Nat. Rev. Genet. 2014, 15, 541-555). The gene delivery agent can be injected directly into the blood, skin, or other place where cells corresponding to the carrier binding specificity are located. The agent can be applied on the skin or mucosa surfaces directly. In that event, it is preferable that the Langerhans' cells are activated on the surface. Activation may be achieved by receptor stimulation (e.g., mannose receptor), toxin activation (cholera toxin), a tissue or cell injury such as inflammation, and may be the consequence of another antigenic stimulation. The gene delivery agent can be infused using a pediatric feeding tube orally, vaginally or rectally in the case of human or animal adults or neonates. Neonates may respond better to oral administration than adults. Alternatively, the gene delivery agent may be packaged in a suppository and inserted in the vagina or rectum.

A person of skill in the art would have the knowledge and experience to determine the biological material needed to complex to the delivery or transfection agent, and the concentrations to successfully transfect cells.

### EXAMPLES

The following Examples of the present disclosure are provided only to further illustrate the exemplary aspects of the present disclosure, and are not intended to limit its scope.

All materials were purchased from Aldrich and were used without further purification, except as noted below. Methylene chloride (CH₂Cl₂) and N,N-dimethylformamide (DMF) were dried using a J.C. Meyer solvent purification system. Deuterated solvents used for NMR spectroscopy were purchased from Cambridge Isotope Laboratories, Inc. Eluents for column chromatography were HPLC grade and purchased from Fisher Scientific. Organic solutions were concentrated by use of a Buchi rotary evaporator. Spectrum Labs dialysis bags were purchased from VWR. All polymerizations were carried out with temperature control via an oil bath under an argon atmosphere in Schlenk flasks.

¹H and ¹³C NMR spectra were recorded in CDCl₃ (except where noted in Experimental Methods) on a Bruker AMX-300, AMX-400, or AMX-500 spectrometer. Data for ¹H NMR are reported as follows: chemical shift in reference to residual CHCl₃ at 7.26 ppm (δ ppm), multiplicity (s=singlet, br s=broad singlet, d=doublet, t=triplet, q=quartet, dd=doublet of doublets, td=triplet of doublets, m=multiplet), coupling constant (Hz), and integration. Data for ¹³C NMR are reported in terms of chemical shift in reference to the CDCl₃ solvent signal (77.16 ppm).

High-resolution mass spectra were obtained from the Columbia University Mass Spectrometry Facility on a JOEL JMSHX110 HF mass spectrometer using FAB+ ionization mode. Thin layer chromatography (TLC) was performed using Teledyne Silica gel 60 F254 plates and viewed under UV light. Flash column chromatography was performed using Teledyne Ultra Pure Silica Gel (230-400 mesh) on a Teledyne Isco Combiflash Rf.

Polyplex size and zeta potential were measured on a Malvern Zetasizer Nano ZS (Malvern, United Kingdom). For all measurements, polyplexes were diluted 1:100 in Milli-Q water at neutral pH. The reported diameters are the average of three measurements, where each measurement comprises at least 10 acquisitions, and the zeta potential was calculated according to the Smoluchowski approximation.

### Example 1

### Procedures for the Synthesis of Bis-1,2-(Diallylamino)-3-Chlorocyclopropenium Chloride

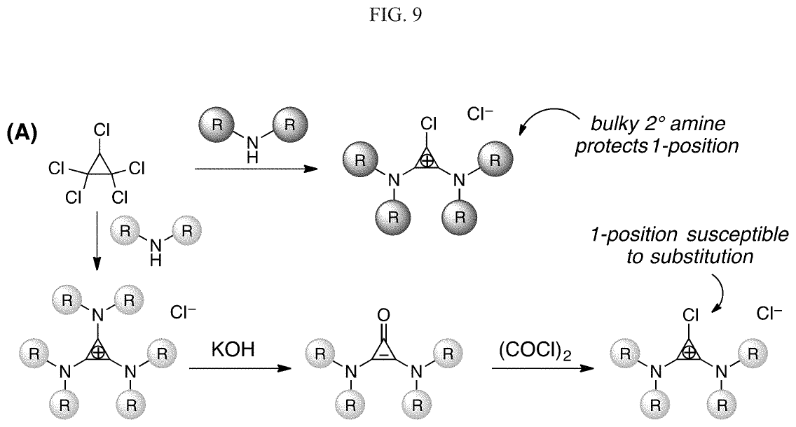

Preparations of BACC1 derivatives have been reported, [30] but briefly, their synthesis involves in situ dehydrochlorination of pentachlorocyclopropane followed by nucleophilic substitution of the resulting tetrachlorocyclopropene with a secondary amine. If the secondary amine is sterically hindered (e.g. Cy and iP), selective double addition yields the desired BACC1 in a single step. However, less sterically demanding amines (e.g. Et, Al, and Mo) lead to the tris(dialkylamino)cyclopropenium products, which require hydrolysis with base to furnish the corresponding cyclopropenone, followed by chlorination with oxalyl chloride (see FIG. 9).

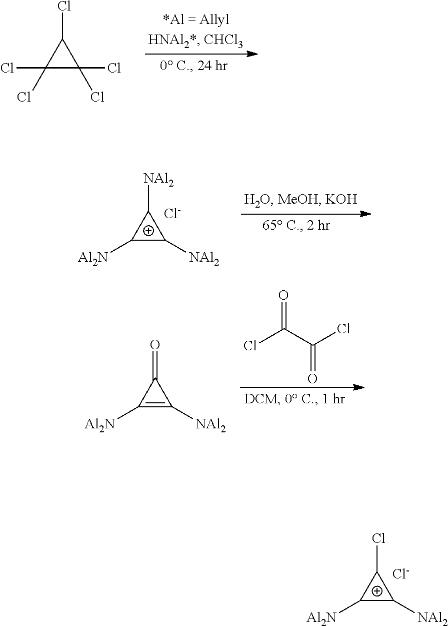

### Synthesis of Bis-2,3-(Diallylamino)-1-Cyclopropenone

This procedure was performed at ambient conditions, without deoxygenation or rigorous efforts to remove water/moisture. Diallylamine (33.0 g, 340 mmol, 7.2 equiv) was slowly added to a solution of pentachlorocyclopropane (10.0 g, 47.2 mmol, 1.0 equiv) in CHCl₃ (400 mL) in a 1 L round bottom flask. The solution turned orange and, after stirring overnight at room temperature, was concentrated in vacuo to yield a crude solid of the same color. A room-temperature solution of water (125 mL), methanol (125 mL), and potassium hydroxide (45 g, 802 mmol) was used to dissolve this solid. The solution was heated to 65° C. and stirred for two hours. Water was removed by rotary evaporation. The resulting solid was dissolved in CH₂Cl₂ and filtered to remove salt. The organic solution was dried with anhydrous sodium sulfate, concentrated in vacuo yielding a crude orange solid. The crude material was purified by silica gel chromatography (20% MeOH in EtOAc) to yield the title product as an orange solid (5.26 g, 21.5 mmol, 46% two-step yield). ¹H NMR (400 MHz, CDCl₃) δ 5.80 (m, 4H, NCH₂CH═CH₂), 5.20 (dd, 8H, NCH₂CH═CH₂), 3.78 (d, 8H, NCH₂CH═CH₂). ¹³C NMR (125 MHz, CDCl₃) δ 133.07, 132.85, 119.83, 118.26, 118.07, 117.85, 53.46. HRMS (FAB+) m/z=245.1654 calcd for C₁₅H₂₀N₂Cl [M⁺H]⁺ 245.16.

### Synthesis of Bis-1,2-(Diallylamino)-3-Chlorocyclopropenium Chloride (BACA1)

Oxalyl chloride (6.86 mL, 79.4 mmol, 2.0 equiv) was slowly added to a solution of bis-2,3-(diallylamino)-1-cyclopropenone (8.9 g, 39.7 mmol, 1.0 equiv) in dry CH₂Cl₂ (250 mL) at 0° C. under argon. The solution was warmed to room temperature and left to react for one hour. The solution was concentrated in vacuo to yield the title product as a dark brown liquid in quantitative yield. ¹H NMR (400 MHz, CDCl₃) δ 5.93 (m, ¹H, NCH₂CH═CH₂), 5.46 (dd, 4H, NCH₂CH═CH₂), 5.30 (dd, 4H, NCH₂CH═CH₂), 4.35 (d, 4H, NCH₂CH═CH₂), 4.10 (d, 4H, NCH₂CH═CH₂).

### Example 2

### Procedures for the Synthesis of Bis-1,2-(Piperidino)-3-Chlorocyclopropenium Chloride or 1,2-Bis(Dibutylamino)-3-Chlorocyclopropenium Chloride

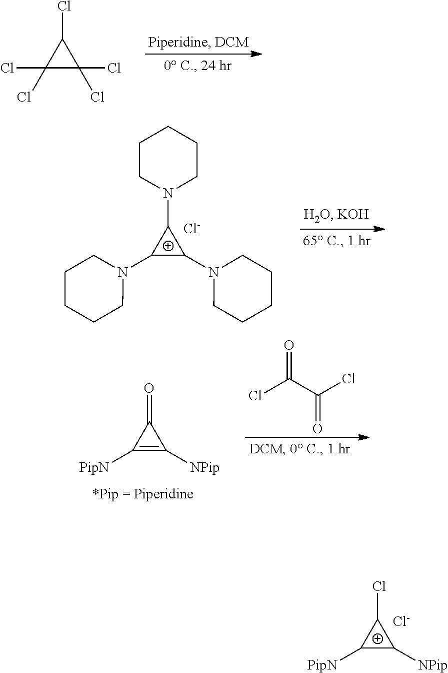

### Synthesis of Bis-2,3-(Piperidino)-1-Cyclopropenone

This procedure was performed at ambient conditions, without deoxygenation or rigorous efforts to remove water/moisture. Piperidine (15.6 g, 0.183 mol, 8 equiv) was slowly added to a solution of pentachlorocyclopropane (5.0 g, 22.8 mmol, 1.0 equiv) in CH₂Cl₂ (230 mL) in a 5000 mL round bottom flask. The solution turned orange and was allowed to stir overnight at room temperature. The reaction mixture was washed with 1M HCl (3×100 mL), deionized (DI) water (1×100 mL), and saturated NaCl solution (1×100 mL), dried over magnesium sulfate, and concentrated in vacuo to yield a crude orange/brown solid. The crude product was dissolved in room temperature DI water (50 mL), and a solution of 10 g potassium hydroxide in 15 mL DI water was added to this mixture. The solution was heated to 65° C. and left to react for one hour. Water was removed by rotary evaporation. The resulting solid was dissolved in CH₂Cl₂ and filtered to remove salt. The organic solution was dried with anhydrous sodium sulfate, concentrated in vacuo yielding a crude orange solid. The crude material was purified by silica gel chromatography (100% EtOAc; 5% MeOH in DCM) to yield the title product as an orange solid (1.465 g, 21.5 mmol, 30% two-step yield). ¹H NMR (400 MHz, CDCl₃) δ 3.28 (s, 8H, C₃(N(CH₂)₂(CH₂)₃)₂), 1.58 (s, 12H, OC₃(N(CH₂)₂(CH₂)₃)₂). ¹³C NMR (100 MHz, CDCl₃) δ 134.85, 120.40, 50.63, 25.42, 23.61. HRMS (FAB+) m/z=221.1647 calcd for C₁₃H₂₀N₂O 220.16 [M+H]⁺ 221.16.

### Synthesis of Bis-1,2-(Piperidino)-3-Chlorocyclopropenium Chloride (BACPip)

Oxalyl chloride (0.41 mL, 4.72 mmol, 2.0 equiv) was slowly added to a solution of bis-2,3-(piperidino)-1-cyclopropenone (0.520 g, 2.36 mmol, 1.0 equiv) in dry CH₂Cl₂ (24 mL) at 0° C. under argon. The solution was warmed to room temperature and left to react for one hour. The solution was concentrated in vacuo to yield the title product as a dark brown liquid in quantitative yield. ¹H NMR (500 MHz, CDCl₃) δ 3.76 (t, 4H, ClC₃(N(CH₂)(CH₂)(CH₂)₃)₂), 3.62 (t, 4H, ClC₃(N(CH₂)(CH₂)(CH₂)₃)₂), 1.88-1.68 (m, 12H, ClC₃(N(CH₂)(CH₂)(CH₂)₃)₂). ¹³C NMR (100 MHz, CDCl₃) δ 132.64, 52.52, 51.32, 24.99, 22.51. HRMS (FAB+) m/z=275.1082 calcd for C₁₅H₂₀N₂Cl [M+H]⁺ 275.11.

### Synthesis of 2,3-Bis(Dibutylamino)-1-Cyclopropenone

This procedure was performed at ambient conditions. Dibutylamine (24.5 g, 190 mmol, 8.0 equiv) was slowly added to a solution of pentachlorocyclopropane (5.0 g, 23.6 mmol, 1.0 equiv) in CH₂Cl₂ (250 mL) in a 1 L round-bottom flask at 0° C. The solution turned orange, and was allowed to warm to room temperature with stirring overnight. The reaction mixture was washed with 1 M HCl (3×100 mL), deionized (DI) water (1×100 mL), and brine (1×100 mL) dried over magnesium sulfate, and concentrated in vacuo to yield a crude orange solid. The solid was dissolved in tert-butanol (50 mL) and to this was added potassium hydroxide (10 g, 178 mmol) in DI water (15 mL). The solution was heated at 70° C. for 2 h, and then water was removed by rotary evaporation. The resulting solid was dissolved in CH₂Cl₂ and filtered to remove salt. The organic solution was dried with anhydrous magnesium sulfate, and concentrated in vacuo to a crude yellow oil. The crude material was purified by silica gel chromatography (100% EtOAc; 5% MeOH in CH₂Cl₂) to yield the title product as an orange solid (2.19 g, 7.08 mmol, 30% two-step yield). ¹H NMR (400 MHz, CDCl₃) δ 3.16 (t, 8H, NCH₂CH₂CH₂CH₃), 1.59 (m, 8H, NCH₂CH₂CH₂CH₃), 1.34 (m, 8H, NCH₂CH₂CH₂CH₃), 0.94 (t, 12H, NCH₂CH₂CH₂CH₃).

### Synthesis of 1,2-Bis(dibutylamino)-3-chlorocyclopropenium Chloride (BACBu)

Oxalyl chloride (0.09 mL, 0.9 mmol, 2.0 equiv) was slowly added to a solution of 2,3-bis(dibutylamino)-1-cyclopropenone (0.150 g, 0.45 mmol, 1.0 equiv) in dry CH₂Cl₂ (5 mL) at 0° C. under argon. The solution was warmed to room temperature and allowed to react for 1 h. The solution was then concentrated in vacuo to yield the title product as a brown liquid in quantitative yield. ¹H NMR (400 MHz, CDCl₃) δ 3.64 (t, 4H, NCH₂CH₂CH₂CH₃), 3.50 (t, 4H, NCH₂CH₂CH₂CH₃), 1.76 (m, 4H, NCH₂CH₂CH₂CH₃), 1.66 (m, 4H, NCH₂CH₂CH₂CH₃), 1.40 (m, 8H, NCH₂CH₂CH₂CH₃), 0.99 (t, 12H, NCH₂CH₂CH₂CH₃).

### Example 3

### Procedures for the Synthesis of Poly Methylamino Styrene (PMAS)

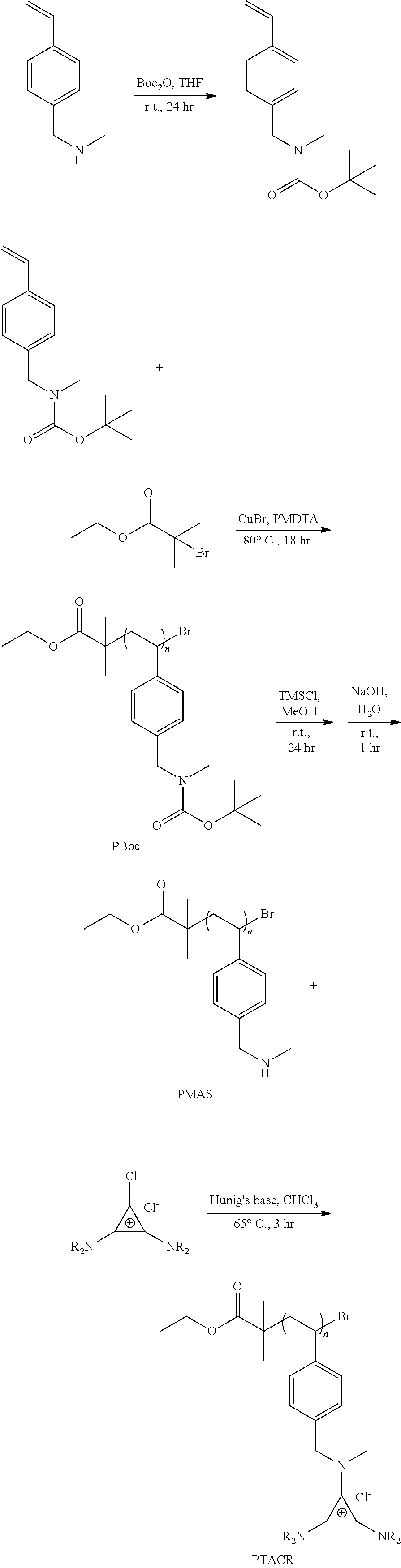

### Synthesis of tert-butyl methyl(4-vinylbenzyl)carbamate

N-methyl-4-vinylbenzylamine (10.07 g, 68.4 mmol, 1 equiv) and THF (300 mL) were added to a 1 L round bottom flask (RBF) and the flask was sealed with a septum secured with copper wire under argon with a gas outlet. Triethylamine (10.4 mL, 74.8 mmol, 1.1 equiv) was added to the RBF, the system was cooled to 0° C., and di-tert-butyl dicarbonate (16.42 g, 74.8 mmol, 1.1 equiv) was slowly injected. The RBF was warmed to room temperature and allowed to stir overnight. The solution was concentrated under vacuum, and the translucent, crude product was dissolved in 300 mL of CH₂Cl₂ and transferred to a 1 L separatory funnel. The solution was washed with 1M HCl (3×100 mL) followed by a single brine wash. The solution was then dried with magnesium sulfate, filtered, and concentrated under vacuum. The crude material was finally purified by silica gel chromatography (100% hexanes then 95% CH₂Cl₂/5% hexanes) to yield the title product as an translucent, colorless liquid (7.22 g, 29.2 mmol, 48%). ¹H NMR (400 MHz, CDCl₃) δ 7.37 (d, 2H, ArH), 7.18 (d, 2H, ArH), 6.71 (dd, 1H, H₂C═CHAr), 5.73 (dd, 1H, H₂C═CHAr), 5.23 (dd, ¹H, H₂C═CHAr), 4.41 (s, 2H, ArCH₂N), 2.81 (s, 3H, NCH₃), 1.48 (s, 9H, NC═OtBuH). ¹³C NMR (125 MHz, CDCl₃) δ 137.7, 136.6, 136.4, 128.0, 127.4, 126.4, 113.7, 79.7, 52.4, 51.7, 33.9, 28.5. HRMS (FAB+) m/z=270.1470 calcd for C₁₅H₂₁N₁O₂ [M+Na]⁺ 270.15.

### Synthesis of PBoc

Copper (I) bromide (10 mg, 7.0E-2 mmol, 0.5 equiv) was added to a dry Schlenk flask and the material was deoxygenated via five vacuum-argon cycles. Degassed N,N,N′,N′,N″-pentamethyldiethylenetriamine (PMDTA) (12.1 mg, 7.0e-2 mmol, 0.5 equiv) was added to the flask and allowed to stir for ten minutes to form Cu complex, a light green mixture. Degassed tert-butyl methyl(4-vinylbenzyl)carbamate (4.5 g, 18.2 mmol, 130 equiv) was then added to the mixture and three freeze-pump-thaw cycles were conducted. The Schlenk flask was closed under argon and degassed ethyl α-bromoisobutyrate (27.3 mg, 0.14 mmol, 1 equiv) was injected, and the reaction mixture was heated to 85° C. and allowed to react for 24 hours. The resulting solution was diluted with methanol and transferred to a 3.5 k MWCO Spectrum Labs dialysis bag and dialyzed against methanol. The resulting solution was concentrated under vacuum to yield a fine, white powder (803.3 mg, 17.9% recovered yield). From SEC: Mn=6700 g mol⁻¹, degree of polymerization ˜65, Ð=1.08. ¹H NMR (400 MHz, CDCl₃) δ 7.15-6.20 (b, 254H, ArH), 4.50-4.21 (b, 130H, ArCH₂N), 2.95-2.56 (b, 198H, NCH₃), 2.01-1.19 (b, 782H, NC═OtBuH, ArCHCH₂).

### Synthesis of PMAS

The PBoc (803.3 mg, 3.25 mmol, 1 eq) was dissolved in methanol (10 mL) in a dry round bottom flask under argon. The flask was cooled to 0° C. and trimethylsilyl chloride (2.47 g, 22.7 mmol, 7 eq) was added. The reaction solution was allowed to stir at room temperature overnight and concentrated under vacuum to yield a white powder. The powder was then re-dissolved in a 1M solution of KOH in methanol and allowed to stir for one hour. The solution was concentrated so that a minimal amount of methanol remained. To this thickened liquid, water was added until white flecks of polymer began to precipitate out. The solution was filtered and the solid white flecks were redissolved in methanol and the previous step was repeated. The resulting polymer was dried under vacuum, yielding a fluffy, white powder (440 mg, 93.6% yield). ¹H NMR (400 MHz, CDCl₃) δ 7.23-6.25 (b, 268H, ArH), 3.77-3.43 (b, 130H, ArCH₂N), 2.57-2.23 (b, 197H, NCH₃), 1.70-1.19 (b, 205H, ArCHCH₂).

### Synthesis of PMAS(Bu)

The procedure was performed open to the atmosphere. Poly(methylaminostyrene) (PMAS) (50 mg, 0.3 mmol, 1 equiv, DP: 50, Mn: 7400, Mw: 8300, Ð: 1.08), synthesized according to the above procedures, was dissolved in CHCl3 (8 mL) in a scintillation vial equipped with a stir bar. To the vial was added N,N-diisopropylethylamine (115 mg, 0.9 mmol, 3 equiv) and BACBu (160 mg, 0.46 mmol, 1.5 equiv). The reaction mixture was allowed to stir at 65° C. for 3 h. The resulting solution was concentrated in vacuo, dissolved in minimum acetone and precipitated once into ethyl acetate at −78° C. The resulting powder was dissolved in methanol and transferred to a 3.5 k MWCO Spectrum Labs dialysis bag and dialyzed against methanol followed by concentration under vacuum to yield a pale brown powder (60 mg, 42% yield). 1H NMR (500 MHz, CDCl₃) δ 7.18-6.07 (b, 200H, ArH), 4.99-4.39 (b, 100H, ArCH₂N), 3.78-2.91 (b, 575H, NCH₃, NCH₂CH₂CH₂CH₃), 1.98-1.58 (b, 400H, NCH₂CH₂CH₂CH₃) 1.39-1.08 (b, 650H, NCH₂CH₂CH₂CH₃, ArCHCH₂) 1.02-0.63 (b, 600H, NCH₂CH₂CH₂CH₃).

### Example 4

### Procedures for the Synthesis of PTACR Homopolymers (Styrenic Backbone)

### Synthesis of PTACA1

This procedure was performed open to the atmosphere. PMAS (58.2 mg, 0.40 mmol, 1 equiv) was dissolved in chloroform (4 mL) in a scintillation vial. To polymer solution was added N,N-diisopropylethylamine (153 mg, 1.19 mmol, 3 equiv) and allowed to stir for 10 minutes. The BACA1 (169 mg, 0.59 mmol, 1.5 equiv) was dissolved in 3 mL of chloroform and added to the reaction vial. The mixture was allowed to stir at 65° C. for three hours. The resulting solution was concentrated in vacuo, diluted with water, and transferred to a 3.5 k MWCO Spectrum Labs dialysis bag, and dialyzed against water and concentrated by rotary evaporation. The polymer was then dissolved in a minimal amount of acetone and precipitated one time into ethyl acetate at −78° C. and again concentrated under vacuum to yield a brown powder (107 mg, 66% yield). ¹H NMR (400 MHz, CDCl₃) δ 7.38-6.27 (b, 263H, ArH), 5.96-5.60 (b, 260H, NCH₂CH═CH₂), 5.43-5.04 (b, 560H, NCH₂CH═CH₂), 4.84-4.42 (b, 130H, ArCH₂N), 4.15-3.80 (b, 513H, NCH₂CH═CH₂), 3.25-2.75 (b, 193H, NCH₃), 2.27-1.03 (b, 195H, ArCHCH₂).

### Synthesis of PTACMo

This procedure was performed open to the atmosphere. PMAS (51.8 mg, 0.39 mmol, 1 equiv) was dissolved in a DMF (3 mL) in a scintillation vial. To the vial was added N,N-diisopropylethylamine (130 mg, 1.02 mmol, 3 equiv), followed by a solution of DCM (3 mL) and BACMo (147 mg, 0.51 mmol, 1.5 equiv). Note: chloroform is not used here, as the resulting PIL is not soluble. DMF can be used as a co-solvent to soluble resultings PILs. The reaction mixture was allowed to stir at 65° C. for three hours. The resulting solution was diluted with water, and transferred to a 3.5 k MWCO Spectrum Labs dialysis bag and dialyzed against water. The resulting solution was concentrated under vacuum to yield a brown powder (126 mg, 91.3% yield). ¹H NMR chemical shifts and integrations have been previously reported for this materials.[31]

### Synthesis of PTACCy

This procedure was performed open to the atmosphere. PMAS (51.3 mg, 0.345 mmol, 1 equiv) was dissolved in chloroform (4 mL) in a scintillation vial. To the vial N,N-diisopropylethylamine (135 mg, 1.04 mmol, 3 equiv) was added, followed by a solution of BACCy (180 mg, 0.38 mmol, 1.1 equiv) in 3 mL chloroform. The reaction mixture was allowed to stir at 65° C. for three hours. The resulting solution was concentrated in vacuo and precipitated twice from DCM into dioxane. The resulting powder was dissolved in methanol and transferred to a 3.5 k MWCO Spectrum Labs dialysis bag. Dialysis was conducted against a 1:1 H₂O: methanol solution. The resulting solution was concentrated under vacuum to yield a light-brown powder (161 mg, 80% yield). 1H NMR chemical shifts and integrations have been previously reported for this materials.[31]

### Synthesis of PTACiP

This procedure was performed open to the atmosphere. PMAS (54 mg, 0.37 mmol, 1 equiv) was dissolved in chloroform (3 mL) in a scintillation vial. To the vial was added N,N-diisopropylethylamine (141 mg, 1.1 mmol, 3 equiv), and a solution of BACiP (202 mg, 0.40 mmol, 1.1 equiv) in 4 mL chloroform was added. The reaction mixture was allowed to stir at 65° C. for three hours. The resulting solution was concentrated in vacuo and precipitated twice from acetone into ethyl acetate at −78° C. (if precipitated polymer does not filter nicely, make the polymer/acetone solution more concentrated). The resulting powder was dissolved in water and transferred to a 3.5 k MWCO Spectrum Labs dialysis bag and dialyzed against water followed by concentration under vacuum to yield a light-brown powder (110 mg, 65% yield). ¹H NMR chemical shifts and integrations have been previously reported for this materials. [31]

### Synthesis of PTACEt

This procedure was performed open to the atmosphere. PMAS (54 mg, 0.368 mmol, 1 equiv) was dissolved in chloroform (3 mL) in a scintillation vial. To the vial was added N,N-diisopropylethylamine (142 mg, 1.1 mmol, 3 equiv) followed by a solution of BACEt (140 mg, 0.55 mmol, 1.1 equiv) in 4 mL chloroform. The reaction mixture was allowed to stir at 65° C. for three hours. The resulting solution was concentrated in vacuo, diluted with water, and transferred to a 3.5 k MWCO Spectrum Labs dialysis bag and dialyzed against water, and subsequently concentrated under vacuum. The polymer was then dissolved in a minimal amount of acetone and precipitated one time into ethyl acetate at −78° C. to yield a brown powder (112 mg, 84% yield). ¹H NMR (400 MHz, CDCl₃) δ 7.30-6.30 (b, 262H, ArH), 4.83-4.54 (b, 130H, ArCH2N), 3.53-3.31 (b, 502H, NCH₂CH₃), 3.26-3.02 (b, 193H, NCH₃), 1.58-1.08 (b, 1009H, NCH₂CH₃, ArCHCH₂).

### Synthesis of PTACPip

This procedure was performed open to the atmosphere. PMAS (50 mg, 0.339 mmol, 1 equiv) was dissolved in chloroform (2 mL) in a scintillation vial. To the vial was added N,N-diisopropylethylamine (0.18 mL, 1.02 mmol, 3 equiv) followed by a solution of BACPip (141 mg, 0.509 mmol, 1.5 equiv) in 6 mL chloroform. The reaction mixture was allowed to stir at 65° C. for three hours. The resulting solution was concentrated in vacuo, diluted with water, and transferred to a 3.5 k MWCO Spectrum Labs dialysis bag and dialyzed against water, and subsequently concentrated under vacuum. The polymer was then dissolved in a minimal amount of acetone and precipitated one time into ethyl acetate at −78° C. to yield a brown powder (129.7 mg, 97% yield). ¹H NMR (400 MHz, acetone-d6) δ 7.90-6.30 (b, 609H, ArH), 5.20-4.60 (b, 274H, ArCH₂N), 3.85-3.45 (b, 1176H, C₃(N(CH₂)₂(CH₂)₂CH₂) 2)), 3.40-3.13 (b, 382H, NCH₃), 1.90-1.37 (b, 1895H) C₃(N(CH₂)₂(CH₂)₂CH₂)₂), ArCHCH₂).

### Example 5

### Procedures for the Synthesis of PS-b-PTACR Block Copolymers

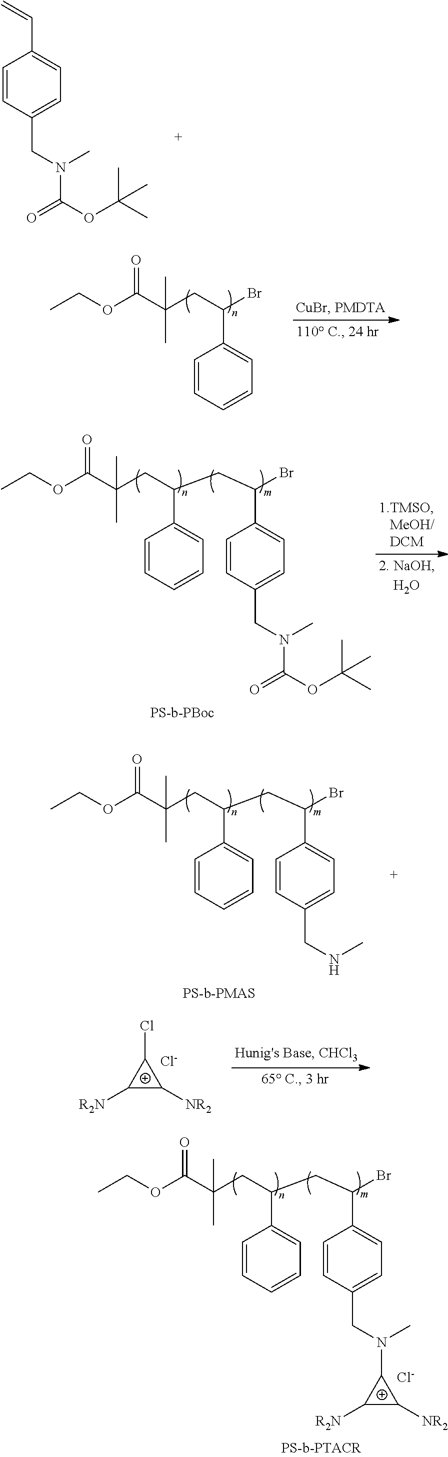

### Synthesis of PS-b-PBoc

Copper (I) bromide (5.5 mg, 3.87e-2 mmol, 0.5 equiv) was added to a dry Schlenk flask and the material was deoxygenated via five vacuum-argon cycles. Degassed N,N,N′,N′,N″-pentamethyldiethylenetriamine (PMDETA) (9.4 mg, 5.4e-2 mmol, 0.7 equiv) was added to the flask and the mixture was stirred for ten minutes, forming a light green mixture. Degassed tert-butyl methyl(4-vinylbenzyl)carbamate (2.6 g, 12 mmol, 150 equiv) was then added to the mixture, and three freeze-pump-thaw cycles were conducted. Lastly, a deoxygenated solution of PS (7 k) (535 mg, 0.77 mmol, 1 equiv) in DMF (1 ml) was injected into the Schlenk flask and the mixture was heated to 85° C. and left to react for 24 hours. The resulting solution was concentrated, dissolved in THF, and then precipitated twice into a 3:1 mixture of methanol-water. The resulting powder was further dried under vacuum, yielding the title product (610 mg, 81% recovered yield). SEC and ¹H NMR reveal block copolymer contains ˜190 units of styrene and ˜127 units Boc-protected monomer. ¹H NMR (400 MHz, CDCl₃) δ 7.15-6.20 (b, 1494H, ArH), 4.50-4.21 (b, 254H, ArCH₂N), 2.95-2.56 (b, 370H, NCH₃), 2.01-1.19 (b, 782H, NC═OtBuH, ArCHCH₂).

### Synthesis of PS-b-PMAS

The PS-b-PBoc (500 mg, 1.27 mmol amine-containing monomer, 1 eq amine monomer) was dissolved in a 50/50 DCM:methanol solution (15 mL) in a round bottom flask under argon. The flask was cooled to 0° C. and trimethylsilyl chloride (2.47 g, 22.7 mmol, 7 eq) was added. The reaction was allowed to stir at room temperature overnight and concentrated under vacuum to yield a white powder. The powder was then re-dissolved in DMSO and 1M NaOH was added dropwise, with stirring, until the polymer precipitated from solution. The resulting slurry was centrifuged, and the supernatant decanted. The polymer was washed two more times with DI water, and collected by centrifugation. The resulting polymer was dried under vacuum, yielding a fluffy, white powder (235.5 mg, 63% yield). ¹H NMR (400 MHz, CDCl₃) δ 7.23-6.25 (b, 268H, ArH), 3.77-3.43 (b, 130H, ArCH₂N), 2.57-2.23 (b, 197H, NCH₃), 1.70-1.19 (b, 205H, ArCHCH₂).

### Synthesis of PS-b-PTACCy

This procedure was performed open to the atmosphere. PS-b-PMAS (56.8 mg polymer, 0.19 mmol amine unit, 1 equiv amine unit) was dissolved in chloroform (6 mL) in a scintillation vial. To the vial was added N,N-diisopropylethylamine (74 mg, 0.57 mmol, 3 equiv) and the BACCy (98.4 mg, 0.21 mmol, 1.1 equiv). The reaction mixture was allowed to stir at 65° C. for three hours. The resulting solution was concentrated in vacuo, diluted with methanol, and transferred to a 3.5 k MWCO Spectrum Labs dialysis bag and dialyzed against methanol. The resulting colloidal solution was concentrated under vacuum to yield a brown powder (130 mg, 91.8% yield). ¹H NMR chemical shifts and integrations have been previously reported for this materials.[31]

### Synthesis of PS-b-PTACiP

This procedure was performed open to the atmosphere. PS-b-PMAS (88 mg polymer, 0.30 mmol amine unit, 1 equiv amine unit) was dissolved in chloroform (7 mL) in a scintillation vial. To the vial was added N,N-diisopropylethylamine (114 mg, 0.89 mmol, 3 equiv) and the BACiP (163 mg, 0.33 mmol, 1.1 equiv). The reaction mixture was allowed to stir at 65° C. for three hours. The resulting solution was concentrated in vacuo, dissolved in water, and transferred to a 3.5 k MWCO Spectrum Labs dialysis bag and dialyzed against a 50/50 mixture of water and methanol. The resulting colloidal solution was concentrated under vacuum to yield a light-brown powder (134 mg, 78.8% yield). ¹H NMR chemical shifts and integrations have been previously reported for this materials.[31]

### Synthesis of PS-b-PTACMo

This procedure was performed open to the atmosphere. PS-b-PMAS (65.7 mg polymer, 0.33 mmol amine unit, 1 equiv amine unit) was dissolved in a DMF (3 mL) in a scintillation vial. To the vial was added N,N-diisopropylethylamine (130 mg, 1.0 mmol, 3 equiv) and a solution of DCM (3 mL) and BACMo (140 mg, 0.50 mmol, 1.5 equiv). The reaction mixture was allowed to stir at 65° C. for three hours. The resulting solution was diluted with water, and transferred to a 3.5 k MWCO Spectrum Labs dialysis bag and dialyzed against a 50/50 mixture of water and methanol. The resulting colloidal solution was concentrated under vacuum to yield a brown powder (157 mg, 93.5% yield). ¹H NMR chemical shifts and integrations have been previously reported for this materials.[31]

### Example 6

### Procedures for the Synthesis of PEO-b-PTACR Block Copolymers

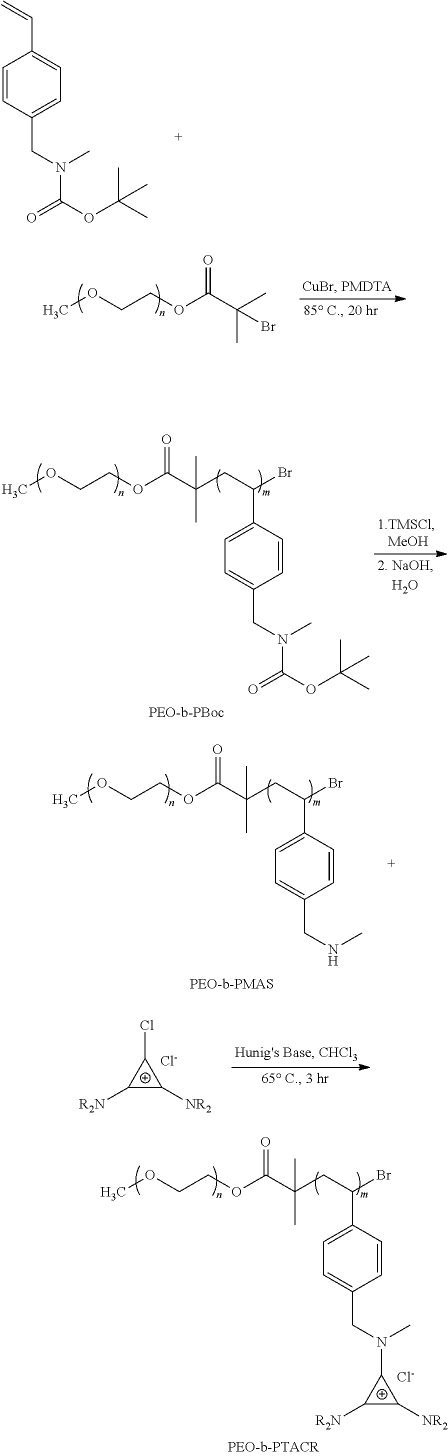

### Synthesis of PEO-b-PBoc

Copper (I) bromide (4.7 mg, 3.28e-2 mmol, 0.5 equiv) was added to a dry schlenk flask and the material was deoxygenated via five vacuum-argon cycles. Sparged N,N,N′,N′,N″-pentamethyldiethylenetriamine (PMDTA) (5.7 mg, 3.82e-2 mmol, 0.5) was added to the flask and let stir for ten minutes, forming a light green mixture. Degassed tert-butyl methyl(4-vinylbenzyl)carbamate (2.23 g, 9 mmol, 150 equiv) was then added to the mixture and three freeze-pump-thaw cycles were conducted. Lastly, a solution of poly(ethylene glycol) methyl ether 2-bromoisobutyrate (300 mg, 6e-2 mmol, 1 equiv) in DMF (1 mL) was injected into the Schlenk flask and the mixture was heated to 85° C. and stirred for 20 hours. The resulting solution was diluted with water and transferred to a 3.5 k MWCO Spectrum Labs dialysis bag and left to dialyze against a solution of 1:1 water-methanol. The resulting solution was concentrated under vacuum to yield a fine, white powder (350 mg, 20% conversion (30 units Boc monomer per chain), 78.7% yield). ¹H NMR (400 MHz, CDCl₃) δ 7.15-6.20 (b, 117H, ArH), 4.50-4.21 (b, 59H, ArCH₂N), 3.72-3.58 (b, 455H, (CH₂CH₂O)114), 2.95-2.56 (b, 89H, NCH₃), 2.01-1.19 (b, 343H, NC═OtBuH, ArCHCH₂).

### Synthesis of PEO-b-PMAS

The PEO-b-PBoc (350 mg polymer, 0.85 mmol Boc unit, 1 equiv Boc unit) was dissolved in methanol (10 mL) in a dry round bottom flask under argon. The flask was cooled to 0° C. and trimethylsilyl chloride (1.25 g, 12.5 mmol, 14.7 equiv) was added. The reaction was allowed to stir at room temperature overnight and then a 0.5M NaOH solution (5 mL) was added to the system. This mixture was stirred for 1 hour and then transferred to a 3.5 k MWCO Spectrum Labs dialysis bag and left to dialyze against water. Finally, the solution was concentrated under vacuum yielding a white powder (191.6 mg, 72% yield). ¹H NMR (400 MHz, MeOD) δ 7.55-6.35 (b, 128H, ArH), 4.35-4.04 (b, 59H, ArCH₂N), 3.72-3.58 (b, 455H, (CH₂CH₂O)114), 2.95-2.56 (b, 91H, NCH₃), 2.01-1.19 (b, 93H, ArCHCH₂).

### Synthesis of PEO-b-PTACCyⁱ

This procedure was performed open to the atmosphere. PEO-b-PMAS (75.9 mg polymer, 0.24 mmol amine unit, 1 equiv amine unit) was dissolved in chloroform (4 mL) in a scintillation vial. N,N-diisopropylethylamine (150 mg, 1.16 mmol, 4.8 equiv) and a solution of BACCy (199 mg, 0.42 mmol, 1.75 equiv) in 3 mL of chloroform were added to the vial. The reaction mixture was allowed to stir at 65° C. for three hours. The resulting solution was concentrated in vacuo and dissolved in methanol before being transferred to a 3.5 k MWCO Spectrum Labs dialysis bag and dialyzed against a 50/50 mixture of water and methanol. The resulting solution was concentrated under vacuum to yield a light-brown powder (118 mg, 64.5% yield). ¹H NMR (400 MHz, CDCl₃) δ 7.35-6.15 (b, 116H, ArH), 5.10-4.60 (b, 54H, ArCH₂N), 3.72-3.58 (b, 455H, (CH₂CH₂O)114), 3.50-2.56 (b, 188H, NCyH, NCH₃), 1.75-(b, 102H, ArCHCH₂).

### Synthesis of PEO-b-PTACiP′

PEO-b-PMAS (50 mg polymer, 0.16 mmol amine unit, 1 equiv amine unit) was dissolved in chloroform (4 mL) in a scintillation vial. To the vial was added N,N-diisopropylethylamine (132 mg, 1.0 mmol, 6 equiv) and the BACiP (117 mg, 0.50 mmol, 3 equiv). The reaction mixture was allowed to stir at 65° C. for three hours. The resulting solution was diluted with water, and transferred to a 3.5 k MWCO Spectrum Labs dialysis bag and dialyzed against water. The resulting solution was concentrated under vacuum to yield a brown powder (71.8 mg, 77% yield). ¹H NMR (500 MHz, CDCl₃) δ 7.40-6.20 (b, 133H, ArH), 5.05-4.50 (b, 60H, ArCH₂N, NCH(CH₃)₂), 4.00-2.90 (b, 630H, NCH(CH₃)₂, (CH2CH2O)114, NCH₃), 2.00-0.80 (b, 805H, ArCHCH₂, NCH(CH₃)₂).

### Synthesis of PEO-b-PTACMoⁱ

This procedure was performed open to the atmosphere. PEO-b-PMAS (65.7 mg polymer, 0.21 mmol amine unit, 1 equiv amine unit) was dissolved in DMF (6 mL) in a scintillation vial. To the vial was added N,N-diisopropylethylamine (130 mg, 1.0 mmol, 5 equiv) and the BACMo (140 mg, 0.50 mmol, 2.4 equiv). The reaction mixture was allowed to stir at 65° C. for three hours. The resulting solution was diluted with water, and transferred to a 3.5 k MWCO Spectrum Labs dialysis bag and dialyzed against water. The resulting solution was concentrated under vacuum to yield a brown powder (106 mg, 90.4% yield). ¹H NMR (400 MHz, MeOD) δ 7.40-6.25 (b, 142H, ArH), 4.85-4.40 (b, 60H, ArCH₂N), 3.85-3.35 (b, 805H, N(CH₂CH₂)₂O, (CH₂CH₂O)114, N(CH₂CH₂)₂O), 3.25-3.00 (b, 90H, NCH₃), 1.70-1.10 (b, 102H, ArCHCH₂).

ⁱFor this reaction, excess of N,N-diisopropylethylamine and the BACC1 ClickabIL was used, although stoichiometric quantities as used in other functionalization reactions would also work.

### Example 7

### Procedures for the Synthesis of PEI-TACR Block Copolymers

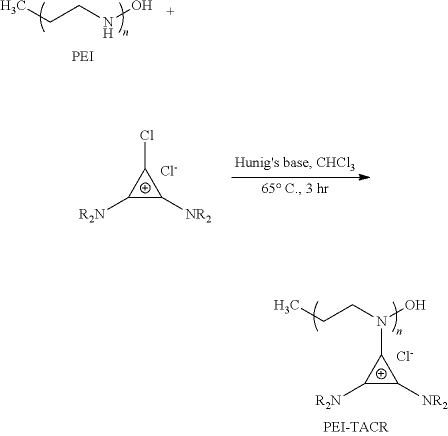

### Synthesis of PEI-Cy

This procedure was performed open to the atmosphere. Linear polyethyleneimine (10 k) (80 mg, 1.86 mmol, 1 equiv) was dissolved in chloroform (7 mL) in a scintillation vial. To the vial was added N,N-diisopropylethylamine (720 mg, 5.57 mmol, 3 equiv) and the BACCy (1.92 g, 4.1 mmol, 2 equiv). The reaction mixture was allowed to stir at 65° C. for 27.5 hours. Note: these reaction conditions require longer time and more equivalents of BACCy than other ClickabIL conditions, potentially because of the steric hindrance of cyclohexyl substituents. The resulting solution was concentrated in vacuo and precipitated twice into dioxane. The resulting powder was dissolved in methanol and transferred to a 3.5 k MWCO Spectrum Labs dialysis bag and dialyzed against methanol. The resulting solution was concentrated under vacuum to yield a yellow-brown powder (220 mg, 25% yield). ¹H NMR (400 MHz, CDCl₃) δ 4.45-3.15 (b, 1810, (CH₂CH₂N)₂₃₃, NCyH), 1.90-0.65 (10165H, NCyH).

### Synthesis of PEI-iP

This procedure was performed open to the atmosphere. Linear polyethyleneimine (10 k) (91 mg, 23 mmol, 1 equiv) was dissolved in chloroform (10 mL) in a scintillation vial. To the vial was added N,N-diisopropylethylamine (901 mg, 79 mmol, 3 equiv) and the BACiP (1.16 g, 26 mmol, 1.1 equiv). The reaction mixture was allowed to stir at 65° C. for three hours. The resulting solution was concentrated in vacuo and then dissolved in water and transferred to a 3.5 k MWCO Spectrum Labs dialysis bag and dialyzed against water. The resulting solution was concentrated under vacuum to yield a yellow-brown powder (394 mg, 60% yield). ¹H NMR (400 MHz, CDCl₃) δ 4.45-3.15 (b, 1860, (CH₂CH₂N)₂₃₃, NCH(CH₃)₂), 1.90-0.65 (5265H, NCH(CH₃)₂).

### Synthesis of PEI-Mo

This procedure was performed open to the atmosphere. Linear polyethyleneimine (10 k) (26.8 mg, 0.623 mmol, 1 equiv) was dissolved in a DMF (3 mL) in a scintillation vial. To the vial was added N,N-diisopropylethylamine (130 mg, 1.02 mmol, 3 equiv) and a solution of DCM (3 mL) and BACMo (251 mg, 0.93 mmol, 1.5 equiv). The reaction mixture was allowed to stir at 65° C. for three hours. The resulting solution was diluted with water, and transferred to a 3.5 k MWCO Spectrum Labs dialysis bag and dialyzed against water. The polymer-water solution was then washed with room temperature chloroform (3×50 mL). The polymer was recovered from rotary evaporation of the aqueous layer to yield a yellow-brown powder (155 mg, 87% yield). ¹H NMR (400 MHz, MeOD) δ 3.95-3.35 (b, 5581H, (CH₂CH₂N—C₃(N(CH₂)₂(CH₂)₂O)₂).

### Synthesis of PEI-Pip

This procedure was performed open to the atmosphere. Linear polyethyleneimine (25 k) (50 mg, 1.16 mmol, 1 equiv) was dissolved in chloroform (4 mL) in a scintillation vial. To the vial was added N,N-diisopropylethylamine (0.61 mL, 3.49 mmol, 3 equiv) and a solution of BACPip (480 mg, 1.74 mmol, 1.5 equiv) in 4 mL chloroform. The reaction mixture was allowed to stir at 65° C. for three hours. The resulting solution was diluted with water, and transferred to a 3.5 k MWCO Spectrum Labs dialysis bag and dialyzed against water. The resulting solution was concentrated under vacuum, and precipitated from acetone into ethyl acetate (−78° C.) and recovered by centrifugation to yield a brown powder (68 mg, 20% recovered yield). ¹H NMR (400 MHz, MeOD) δ 3.95-3.40 (b, 6,972H, ((CH₂CH₂N)—C₃(N(CH₂)₂(CH₂)₂CH₂)₅₈₁) δ 1.90-1.60 (b, 6,972H, ((CH₂CH₂N)—C₃(N(CH₂)₂(CH₂)₂CH₂)₂)₅₈₁).

### Synthesis of PEI(Bu)

This procedure was performed open to the atmosphere. Linear 25 k polyethyleneimine (20 mg, 0.46 mmol, 1 equiv) was dissolved in CHCl₃ (8 mL) in a scintillation vial equipped with a stir bar. To the vial was added N,N-diisopropylethylamine (180 mg, 1.4 mmol, 3 equiv) and BACBu (250 mg, 0.70 mmol, 1.5 equiv). The reaction mixture was allowed to stir at 65° C. for 3 h. The resulting solution was concentrated in vacuo and precipitated once into ethyl acetate at −78° C. The resulting powder was dissolved in methanol and transferred to a 3.5 k MWCO Spectrum Labs dialysis bag and dialyzed against methanol. The resulting solution was concentrated vacuum to yield a yellow-brown powder (25 mg, 15% yield). ¹H NMR (400 MHz, CDCl₃) δ 4.45-3.15 (b, 7000H, CH₂CH₂N)₅₈₁, NCH₂CH₂CH₂CH₃) 1.71-1.55 (b, 3500H, NCH₂CH₂CH₂CH₃), 1.43-1.22 (b, 5400H, NCH₂CH₂CH₂CH₃), 1.00-0.90 (b, 6200H, NCH₂CH₂CH₂CH₃).

### Example 8

### Information for Biological Experiments

### Cell Culture

HEK 293T cells (American Type Culture Collection) were grown in Dulbecco's Modified Eagle Medium with L-glutamine (Gibco) supplemented with 10% FBS (Atlanta Biologicals) and 1% penicillin/streptomycin (Gibco). Cultures were incubated in humidified tissue incubators (Thermo Scientific) at 37° C. and 5% CO₂.

### Cell Viability Measurements

Trypan blue dye exclusion counting was performed in triplicate with an automated cell counter (ViCell, Beckman-Coulter). Cell viability under experimental conditions is reported as a percentage relative to untreated cells.

### Polymer-DNA Complexation

Solutions of polymer in RNase-free water were added to 3 μg of pDNA (gWiz-Luciferase, Aldevron, Fargo, N. Dak.) at specified loadings. The solutions were then vortexed at 1500 rpm for 3 min at room temperature.

### Cell Transfection and Luciferase Expression

293T cells were seeded on 12-well plates at a density of 50,000 cells per well 24 hours prior to transfection. The media was then evacuated, replaced with fresh media, and supplemented with the polymer-pDNA complex. After 48 hours of incubation, cell viability was measured, and cells were re-plated on 96-well plates and analyzed for luciferase activity according to manufacturer's protocol. Briefly, cells were rinsed with PBS and lysed with 20 μL/well 1× Cell Lysis Buffer (Promega, Madison, Wis.). To the cell lysates was added 100 μL/well of Luciferase Assay Reagent (Promega) and the light produced was measured on a plate reader (PerkinElmer, Waltham, Mass.). Results were expressed as relative light units (RLU) normalized to cell counts, with error bars representing the standard deviation from the triplicate measurement.

### Charge Ratio Calculation:

$$\frac{( 3 μ g pDNA ) \times ( MW per polymer repeat unit )}{330 g per pDNA nucleotide} = Mass of polymer required for 1 \text{:} 1 charge ratio with pDNA$$

### Hydrodynamic Size and Zeta Potential Measurement

Polyplex size and zeta potential were measured on a Malvern Zetasizer Nano ZS (Malvern Instruments, Malvern, UK). For all measurements, polyplexes were diluted 1:100 in Milli-Q water at neutral pH. The reported diameters are the average of three measurements, where each measurement comprises at least 10 acquisitions, and the zeta potential was calculated according to the Smoluchowski approximation.

### Gel Electrophoresis Shift Assay

Polyplexes were prepared at different weight ratios by adding 10 μL of polymer in Milli-QH2O to 10 μL of pDNA (5 ng/μL), and vortexing at 1500 rpm for 3 min at room temperature. To the polyplex solution was then added 2 μL of loading dye, for a total volume of 22 μL, which was subsequently added to the well. Agarose gels were prepared as 1 wt % in tris-acetate EDTA (TAE) buffer with 2 μL ethidium bromide and run at 100 V for 20 min. Gels were visualized under UV illumination at 365 nm

The following references are incorporated herein in their entireties:

### REFERENCES

[Ref. 1] a) C. J. Hawker, K. L. Wooley, Science 2005, 309, 1200-1205; b) K. Matyjaszewski, N. V. Tsarevsky, Nat. Chem. 2009, 1, 276-288.
[Ref. 2] a) T. P. Lodge, Science 2008, 321, 50-51; b) J. S. Bandar, A. Barthelme, A. Y. Mazori, T. H. Lambert, Chem. Sci. 2015, 6, 1537-1547.
[Ref. 3] a) J. Ramos, J. Forcada, R. Hidalgo-Alvarez, Chem. Rev. 2014, 114, 367-428; b) H. Yin, R. L. Kanasty, A. A. Eltoukhy, A. J. Vegas, J. R. Dorkin, D. G. Anderson, Nat. Rev. Genet. 2014, 15, 541-555.
[Ref. 4] a) J. Pan, C. Chen, L. Zhuang, J. Lu, Acc. Chem. Res. 2011, 45, 473-481; b) B. Qiu, B. Lin, L. Qiu, F. Yan, J. Mater. Chem. 2012, 22, 1040-1045.
[Ref. 5] Y. Wang, K. Kimura, P. L. Dubin, W. Jaeger, Macromolecules 2000, 33, 3324-3331.
[Ref. 6] a) J. N. Hunt, K. E. Feldman, N. A. Lynd, J. Deek, L. M. Campos, J. M. Spruell, B. M. Hernandez, E. J. Kramer, C. J. Hawker, Adv. Mater. 2011, 23, 2327-2331; b) J. Fang, B. H. Wallikewitz, F. Gao, G. Tu, C. Mueller, G. Pace, R. H. Friend, W. T. S. Huck, J. Am. Chem. Soc. 2010, 133, 683-685; c) J. Pan, C. Chen, L. Zhuang, J. Lu, Acc. Chem. Res. 2012, 45, 473-481.
[Ref. 7] C. E. Sing, J. W. Zwanikken, M. Olvera de la Cruz, Nat. Mater. 2014, 13, 694-698.
[Ref. 8] a) A. Zintchenko, A. Philipp, A. Dehshahri, E. Wagner, Bioconjugate Chem. 2008, 19, 1448-1455; b) H. L. Jiang, J. T. Kwon, Y. K. Kim, E. M. Kim, R. Arote, H. J. Jeong, J. W. Nah, Y. J. Choi, T. Akaike, M. H. Cho, C. S. Cho, Gene Ther. 2007, 14, 1389-1398; c) F. M. Kievit, O. Veiseh, N. Bhattarai, C. Fang, J. W. Gunn, D. Lee, R. G. Ellenbogen, J. M. Olson, M. Zhang, Adv. Funct. Mater. 2009, 19, 2244-2251; d) S. K. Samal, M. Dash, S. Van Vlierberghe, D. L. Kaplan, E. Chiellini, C. van Blitterswijk, L. Moroni, P. Dubruel, Chem. Soc. Rev. 2012, 41, 7147-7194.
[Ref. 9] a) Y. Jiang, J. L. Freyer, P. Cotanda, S. D. Brucks, K. L. Killops, J. S. Bandar, C. Torsitano, N. P. Balsara, T. H. Lambert, L. M. Campos, Nat. Commun. 2015, 6; b)

J. S. Bandar, T. H. Lambert, Synthesis 2013, 45, 2485-2498.
[Ref. 10] K. M. Hugar, H. A. Kostalik, G. W. Coates, J. Am. Chem. Soc. 2015, 137, 8730-8737.
[Ref. 11] a) Y.-C. Tseng, S. Mozumdar, L. Huang, Adv. Drug Deliv. Rev. 2009, 61, 721-731; b) A. Schroeder, C. G. Levins, C. Cortez, R. Langer, D. G. Anderson, J. Intern. Med. 2010, 267, 9-21; c) H. Yoon, E. J. Dell, J. L. Freyer, L. M. Campos, W.-D. Jang, Polymer 2014, 55, 453-464.
[Ref. 12] a) I. Yudovin-Farber, C. Yanay, T. Azzam, M. Linial, A. J. Domb, Bioconjugate Chem. 2005, 16, 1196-1203; b) T. Kawano, T. Okuda, H. Aoyagi, T. Niidome, J. Control. Release 2004, 99, 329-337.
[Ref. 13] M. Li, S. Schlesiger, S. K. Knauer, C. Schmuck, Angew. Chem., Int. Ed. 2015, 54, 2941-2944.
[Ref. 14] S. De Smedt, J. Demeester, W. Hennink, Pharm. Res. 2000, 17, 113-126.
[Ref. 15] a) R. Weiss, T. Brenner, F. Hampel, A. Wolski, Angew. Chem., Int. Ed. Engl. 1995, 34, 439-441; b) R. Weiss, M. Rechinger, F. Hampel, A. Wolski, Angew. Chem., Int. Ed. Engl. 1995, 34, 441-443.
[Ref. 16] a) R. A. Moss, S. Shen, K. Krogh-Jespersen, J. A. Potenza, H. J. Schugar, R. C. Munjal, J. Am. Chem. Soc. 1986, 108, 134-140; b) K. L. Killops, S. D. Brucks, K. L. Rutkowski, J. L. Freyer, Y. Jiang, E. R. Valdes, L. M. Campos, Macromolecules 2015, 48, 2519-2525.
[Ref. 17] R. K. Iha, K. L. Wooley, A. M. Nystrom, D. J. Burke, M. J. Kade, C. J. Hawker, Chem. Rev. 2009, 109, 5620-5686.
[Ref. 18] a) A. Laschew sky, Curr. Opin. Colloid Interface Sci. 2012, 17, 56-63; b) P. Espeel, F. E. Du Prez, Macromolecules 2015, 48, 2-14.
[Ref. 19] U. H. Choi, M. Lee, S. Wang, W. Liu, K. I. Winey, H. W. Gibson, R. H. Colby, Macromolecules 2012, 45, 3974-3985.
[Ref. 20] a) M. Singh, O. Odusanya, G. M. Wilmes, H. B. Eitouni, E. D. Gomez, A. J. Patel, V. L. Chen, M. J. Park, P. Fragouli, H. Iatrou, N. Hadjichristidis, D. Cookson, N. P. Balsara, Macromolecules 2007, 40, 4578-4585; b) R. L. Weber, Y. Ye, A. L. Schmitt, S. M. Banik, Y. A. Elabd, M. K. Mahanthappa, Macromolecules 2011, 44, 5727-5735.
[Ref. 21] a) 0. J. Curnow, M. T. Holmes, L. C. Ratten, K. J. Walst, R. Yunis, RSCs Adv. 2012, 2, 10794-10797; b) 0. J. Curnow, D. R. MacFarlane, K. J. Walst, Chem. Comm. 2011, 47, 10248-10250.
[Ref. 22] a) J.-H. Choi, Y. Ye, Y. A. Elabd, K. I. Winey, Macromolecules 2013, 46, 5290-5300; b) G. Sudre, S. Inceoglu, P. Cotanda, N. P. Balsara, Macromolecules 2013, 46, 1519-1527.
[Ref. 23] C. Barner-Kowollik, F. E. Du Prez, P. Espeel, C. J. Hawker, T. Junkers, H. Schlaad, W. Van Camp, Angew. Chem., Int. Ed. 2011, 50, 60-62.
[Ref. 24] T. Janoschka, A. Teichler, A. Krieg, M. D. Hager, U. S. Schubert, J. Polym. Sci., Part A: Polym. Chem. 2012, 50, 1394-1407.
[Ref. 25] a) J. Yuan, D. Mecerreyes, M. Antoniett i, Prog. Polym. Sci. 2013, 38, 1009-1036; b) A. Saha, S. De, M. C. Stuparu, A. Khan, J. Am. Chem. Soc. 2012, 134, 17291-17297.
[Ref. 26] a) O. Boussif, F. Lezoualc'h, M. A. Zanta, M. D. Mergny, D. Scherman, B. Demeneix, J. P. Behr, Proc. Natl. Acad. Sci. 1995, 92, 7297-7301; b) B. I. Florea, C. Meaney, H. E. Junginger, G. Borchard, AAPS PharmSci 2002, 4, 1-11.
[Ref. 27] S. M. Moghimi, P. Symonds, J. C. Murray, A. C. Hunter, G. Debska, A. Szewczyk, Mol. Ther. 2005, 11, 990-995.
[Ref. 28] a) H. Y. Kuchelmeister, S. Karczewski, A. Gutschmidt, S. Knauer, C. Schmuck, Angew. Chem., Int. Ed. 2013, 52, 14016-14020; b) T. Yu, X. Liu, A.-L. Bolcato-Bellemin, Y. Wang, C. Liu, P. Erbacher, F. Qu, P. Rocchi, J.-P. Behr, L. Peng, Angew. Chem., Int. Ed. 2012, 51, 8478-8484; c) C. Buerkli, S. H. Lee, E. Moroz, M. C. Stuparu, J.-C. Leroux, A. Khan, Biomacromolecules 2014, 15, 1707-1715.
[Ref. 29] J. Zhou, J. Liu, C. J. Cheng, T. R. Patel, C. E. Weller, J. M. Piepmeier, Z. Jiang, W. M. Saltzman, Nat. Mater. 2012, 11, 82-90.
[Ref. 30] See references discussed in: Bandar, J. S.; Lambert, T. H. Synthesis 2013, 45, 2485.
[Ref. 31] Jiang, Y.; Freyer, J. L.; Cotanda, P.; Brucks, S. D.; Killops, K. L.; Bandar, J. S.; Torsitano, C.; Balsara, N. P.; Lambert, T. H.; Campos, L. M. Nat. Commun. 2015, 6.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A non-viral gene delivery agent, comprising:
a poly amine comprising a polymer containing a secondary amine; and
a derivative of a cyclopropenium ion,
wherein the polyamine and the derivative of the cyclopropenium ion are covalently attached.

2. The non-viral gene delivery agent of claim 1, wherein the derivative is a bis(dialkylamino)cyclopropenium chloride salt.

3. The non-viral gene delivery agent of claim 1, wherein the secondary amine is a dialkylamino group.

4. The non-viral gene delivery agent of claim 3, where in the dialkylamino group is selected from the group consisting of: dicyclohexylamine, diisopropylamine, morpholine, piperidine, diethylamine, diallylamine, and dibutylamine, and combinations thereof.

5. The non-viral gene delivery agent of claim 1, wherein the polyamine is a linear polyethyleneimine or a linear polyvinylbenzylmethylamine or poly(methylaminostyrene).

6. The non-viral gene delivery agent of claim 1, wherein the derivative of the cyclopropenium ion is bis(dialkylamino)cyclopropenium chloride (BACC1) ionic liquid.

7. The non-viral gene delivery agent of claim 1, wherein the agent is selected from the group consisting of: poly(methylaminostyrene)(piperidine), poly(methylaminostyrenexmorpholine), polyethyleneimine(piperidine), poly(methylaminostyrene)(«-butyl), poly(methylaminostyrene)(isopropyl), polyethyleneimine(«-butyl), polyethyleneimine(isopropyl) and polyethyleneimine(morpholine).

8. (canceled)

9. A non-viral transfection agent, comprising: 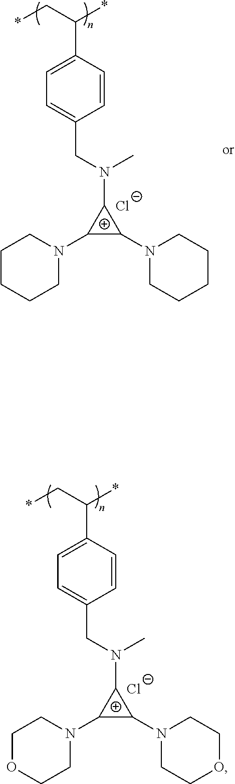
wherein n=100-200.

10. The non-viral transfection agent of claim 9, wherein n=130-140.

11. (canceled)

12. A non-viral transfection agent of: 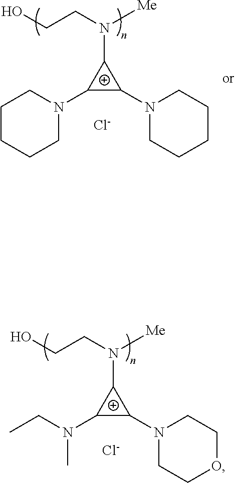
wherein n=220-590.

13. The non-viral transfection agent of claim 12, wherein n=575-585.

14. (canceled)

15. The non-viral gene delivery agent, comprising poly(methylaminostyrene)(«-butyl) (PMAS(Bu)):

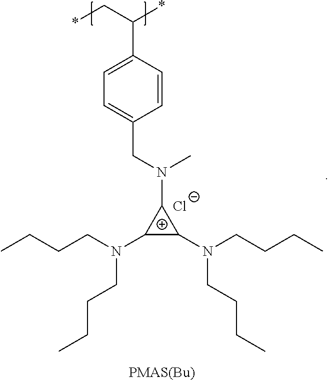

16. A method of transfecting a cell with a genetic material, comprising:
complexing a non-viral gene delivery agent with the genetic material to form a polyplex;
administering the polyplex to a candidate set of cells for transfection; and
transfecting the cells with the polyplex,
wherein the non-viral gene delivery agent comprises a poly amine comprising a polymer containing a secondary amine, wherein the polyamine and a derivative of a cyclopropenium ion are covalently attached.

17. The method of claim 16, wherein the transfection agent is selected from the group consisting of: Poly(methylaminostyrene)(piperidine), poly(methylaminostyrenexmorpholine), polyethyleneimine(piperidine), poly(methylaminostyrene(«-butyl), poly(methylaminostyrene)(isopropyl), polyethyleneimine(«-butyl), polyethyleneimine(isopropyl) and polyethyleneimine(morpholine).

18. A method of synthesis of a non-viral delivery agent, comprising:
polymerizing a polymer precursor backbone, wherein the polymer precursor backbone is a polyamine comprising a polymer containing a secondary amine;
polymerizing a bis(dialkylamino)cyclopropenium chloride derivative (BACC1); and covalently attaching the B ACC1 to the polymer precursor backbone by a click reaction,
wherein the non-viral delivery agent produced by the click reaction is a trisaminocyclopropenium (TAC) polymer, and
wherein the TAC polymer comprises a polyamine comprising a polymer containing a secondary amine, wherein the polyamine and a derivative of a cyclopropenium ion are covalently attached.

19. A complex between
a) a poly amine comprising a polymer containing a secondary amine; and a derivative of a cyclopropenium ion, wherein the polyamine and the derivative of the cyclopropenium ion are covalently attached, and
b) a gene.

20. The complex of claim 19, comprising: 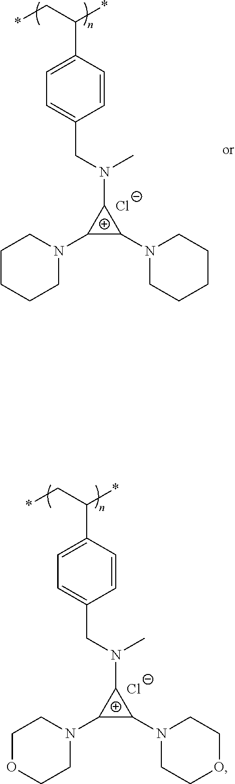
wherein n=100-200.

21. The complex of claim 19, comprising: 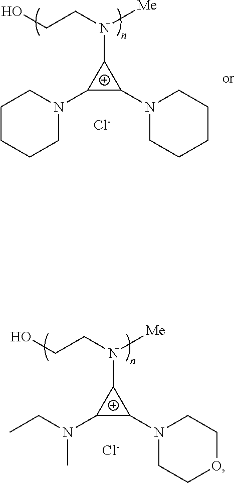
wherein n=220-590.

22. The complex of claim 19, comprising poly(methylaminostyrene)(s-butyl) (PMAS(Bu)): [chemical structure]
